SEQUENCE LISTING (14 sequences)

SEQ ID NO: 1 - protein, 16 aa, Artificial Sequence
  Description of Artificial Sequence Synthetic peptide
CDLMQRERGKLDGQDR

SEQ ID NO: 2 - protein, 11 aa, Artificial Sequence
  Description of Artificial Sequence Synthetic peptide
AFGGQSLKFGK

SEQ ID NO: 3 - protein, 9 aa, Artificial Sequence
  Description of Artificial Sequence Synthetic peptide
FGKGGQAHR

SEQ ID NO: 4 - protein, 13 aa, Artificial Sequence
  Description of Artificial Sequence Synthetic peptide
AKNTVIATGGYGR

SEQ ID NO: 5 - protein, 11 aa, Artificial Sequence
  Description of Artificial Sequence Synthetic peptide
YAPVAKDLASR

SEQ ID NO: 6 - protein, 20 aa, Artificial Sequence
  Description of Artificial Sequence Synthetic peptide
ACALSIAESCRPGDKVPSIK

SEQ ID NO: 7 - protein, 18 aa, Artificial Sequence
  Description of Artificial Sequence Synthetic peptide
VPSIKANAGEESVMNLDK

SEQ ID NO: 8 - protein, 15 aa, Artificial Sequence
  Description of Artificial Sequence Synthetic peptide
ANAGEESVMNLDKLR

SEQ ID NO: 9 - protein, 12 aa, Artificial Sequence
  Description of Artificial Sequence Synthetic peptide
ISQLYGDLKHLK

SEQ ID NO: 10 - protein, 7 aa, Artificial Sequence
  Description of Artificial Sequence Synthetic peptide
HLKTFDR

SEQ ID NO: 11 - protein, 6 aa, Artificial Sequence
  Description of Artificial Sequence Synthetic peptide
EDYKVR

SEQ ID NO: 12 - protein, 15 aa, Artificial Sequence
  Description of Artificial Sequence Synthetic peptide
VDEYDYSKPIQGQQK

SEQ ID NO: 13 - protein, 18 aa, Artificial Sequence
  Description of Artificial Sequence Synthetic peptide
KPFGEHWRKHTLSYVDIK

SEQ ID NO: 14 - protein, 12 aa, Artificial Sequence
  Description of Artificial Sequence Synthetic peptide
HTLSYVDIKTGK

(19) United States
(12) Patent Application Publication
(10) Pub. No.: US 2013/0171125 A1
(43) Pub. Date: Jul. 4, 2013

(54) Methods for the Regulation of Cellular Metabolism Through the Modulation of SIRT3 Activity

(71) Applicants: Marcia C. Haigis, Winchester, MA (US); Lydia Finley, Boston, MA (US)

(72) Inventors: Marcia C. Haigis, Winchester, MA (US); Lydia Finley, Boston, MA (US)

(73) Assignee: National Institutes of Health (NIH), U.S. Dept. of Health and Human Services (DDS), U.S. Government, Bethesda, MD (US)

(21) Appl. No.: 13/697,708

(22) PCT Filed: May 11, 2011
(86) PCT No.: PCT/US2011/036084

§ 371 Date: Feb. 21, 2013

Related U.S. Application Data
(60) Provisional application No. 61/333,487, filed on May 11, 2010.

(51) Int. Cl. G01N 33/68; C12Q 1/68; C12N 5/09

(52) U.S. Cl. CPC G01N 33/6872; C12N 5/0693; C12Q 1/6886
USPC 424/94.6; 435/375; 435/7.4; 435/6.12; 435/6.11

(57) ABSTRACT

The present invention relates to methods of preventing or treating cancer and/or decreasing damage to organs or tissues exposed to hypoxic conditions through the use of agents that modulate the expression or activity of SIRT3.

### RELATED APPLICATIONS

This application claims the benefit of priority to U.S. Provisional Patent Application Ser. No. 61/333,487, filed May 11, 2010; which is hereby incorporated by reference in its entirety.

### GOVERNMENT INTEREST

This invention was made with Government support under National Institutes of Health Grant AG032375. The Government has certain rights in the invention.

### BACKGROUND OF THE INVENTION

Cells that alter their metabolic profile to undergo elevated levels of glycolysis have a survival advantage when subjected to hypoxic environments, such as those found in solid tumors or blood-deprived tissues.

Cancer cells often preferentially perform glycolysis in order to rapidly synthesize the biomass essential for their growth in a hypoxic environment. The highly glycolytic phenotype of cancer cells is used to diagnose and monitor the growth of tumors using positron emission tomography (PET) technology.

The switch from oxidative to glycolytic metabolism is therefore a hallmark of tumorigenesis. This reprogramming of cell metabolism, known as the Warburg effect, is critical to cancer initiation and progression, yet the regulatory network underlying this metabolic reprogramming has not been elucidated. Methods that reduce the level of glycolysis in cancer cells are therefore useful in the treatment of tumors that have undergone metabolic reprogramming.

Additionally, hypoxic conditions also arise when tissues or organs have impaired access to the blood supply, such as occurs during stroke, myocardial infarction or peripheral vascular disease. In such situations, the level of tissue damage would be reduced if the blood-deprived cells underwent elevated levels of glycolytic metabolism. Methods that activate glycolytic metabolism are therefore useful for the reduction of tissue damage following such cardiovascular events.

Methods of modulating the level of glycolytic metabolism in a cell therefore offer great promise for the prevention and/or treatment of cancers that have altered metabolic reprogramming and for the reduction of tissue damage during cardiovascular disease.

### SUMMARY OF THE INVENTION

In certain embodiments, the present invention relates to a method of reducing glycolysis in a cell comprising contacting said cell with an agent that increases the activity or expression of SIRT3 in said cell. In some embodiments, the agent is a small molecule, a polypeptide, a SIRT3 protein, a SIRT3 protein fragment or a polynucleotide, including a polynucleotide that encodes a SIRT3 protein. In some embodiments the cell is a cancer cell, including cancer cells that have a highly glycolytic phenotype and/or that express elevated levels of HIF1α.

In some embodiments, the present invention relates to a method of increasing glycolysis in a cell comprising contacting said cell with an agent that decreases the activity or expression of SIRT3 in said cell. In certain embodiments the agent is a small molecule, a peptide that inhibits the activity of SIRT3 or an inhibitory polynucleotide, including siRNA, shRNA, antisense RNA molecules, and polynucleotides that encode an siRNA, shRNA, and/or an antisense RNA molecule. In some embodiments, the cell has been exposed to a hypoxic environment. In certain embodiments the cell is a neuron, a cardiac myocyte or a skeletal myocyte.

In certain embodiments, the present invention relates to a method of reducing proliferation or survival of a cell under hypoxic conditions comprising contacting said cell with an agent that increases the activity or expression of SIRT3 in said cell. In some embodiments, the agent is a small molecule, a polypeptide, a SIRT3 protein, a SIRT3 protein fragment or a polynucleotide, including a polynucleotide that encodes a SIRT3 protein. In some embodiments the cell is a cancer cell, including cancer cells that have a highly glycolytic phenotype and/or that express elevated levels of HIF1α.

In some embodiments, the present invention relates to a method of increasing proliferation or survival of a cell under hypoxic conditions comprising contacting said cell with an agent that decreases the activity or expression of SIRT3 in said cell. In certain embodiments the agent is a small molecule, a peptide that inhibits the activity of SIRT3 or an inhibitory polynucleotide, including siRNA, shRNA, antisense RNA molecules, and polynucleotides that encode an siRNA, shRNA, and/or an antisense RNA molecule. In some embodiments, the cell has been exposed to a hypoxic environment. In certain embodiments the cell is a neuron, a cardiac myocyte or a skeletal myocyte.

In some embodiments, the present invention relates a method of treating or preventing cancer in a subject comprising administering to said subject an effective dose of an agent that that increases the activity or expression of SIRT3. In some embodiments, the agent is a small molecule, a polypeptide, a SIRT3 protein, a SIRT3 protein fragment or a polynucleotide, including a polynucleotide that encodes a SIRT3 protein. In certain embodiments the cancer is a solid tumor and/or a metastatic tumor. In certain embodiments the tumor has a highly glycolytic phenotype and/or expresses elevated levels of HIF1α.

In certain embodiments the present invention relates to a method of treating or preventing damage to a tissue or organ in a subject comprising administering to said subject an effective dose of an agent that decreases the activity or expression of SIRT3, wherein said tissue or organ is exposed to hypoxia. In certain embodiments the agent is a small molecule, a peptide that inhibits the activity of SIRT3 or an inhibitory polynucleotide, including siRNA, shRNA, antisense RNA molecules, and polynucleotides that encode an siRNA, shRNA, and/or an antisense RNA molecule. In some embodiments the subject has had a stroke, a myocardial infarction or peripheral vascular disease.

In some embodiments, the present invention relates a method of reducing the growth of a tumor in a subject comprising administering to said subject an effective dose of an agent that that increases the activity or expression of SIRT3. In some embodiments, the agent is a small molecule, a polypeptide, a SIRT3 protein, a SIRT3 protein fragment or a polynucleotide, including a polynucleotide that encodes a SIRT3 protein. In certain embodiments the tumor is a solid tumor and/or a metastatic tumor. In certain embodiments the tumor has a highly glycolytic phenotype and/or expresses elevated levels of HIF1α.

In certain embodiments the present invention relates to a method of reducing the damage caused by a stroke, a myocardial infarction, or peripheral vascular disease comprising administering to said subject an effective dose of an agent that decreases the activity or expression of SIRT3, wherein said tissue or organ is exposed to hypoxia. In certain embodiments the agent is a small molecule, a peptide that inhibits the activity of SIRT3 or an inhibitory polynucleotide, including siRNA, shRNA, antisense RNA molecules, and polynucleotides that encode an siRNA, shRNA, and/or an antisense RNA molecule.

In some embodiments the present invention relates to a method of determining the metabolic phenotype of a tumor cell. In some embodiments, the method includes the steps of providing a tumor cell and determining the level of SIRT3 protein or RNA in the tumor cell. In certain embodiments, a decreased level of SIRT3 protein or RNA in the tumor cell relative to the level of SIRT3 protein or RNA in a non-tumor cell indicates that the tumor cell has a glycolytic phenotype. The level of SIRT3 RNA in the tumor cell may be determined, for example using a nucleic acid probe (e.g., a nucleic acid probe directly or indirectly labelled with a detectable moiety) that specifically binds to SIRT3 RNA. The level of SIRT3 protein in the tumor cell may be determined, for example, using an antibody or antigen binding fragment thereof that specifically binds to SIRT3 protein (e.g., an antibody or antigen binding fragment thereof directly or indirectly labelled with a detectable moiety). In some embodiments, the tumor cell is of the same tissue type as the non-tumor cell. In some embodiments, the method also includes the step of determining the level of SIRT protein or RNA in the non-tumor cell. In certain embodiments, the method also includes the step of obtaining the tumor cell and/or the non-tumor cell from a patient. For example, in some embodiments, the tumor cell and the non-tumor cell may be obtained from the same patient. In some embodiments, the method also includes the step of administering to the patient an agent that increases the expression or activity of SIRT3. In some embodiments, the agent is administered if the SIRT3 protein or RNA level in the tumor cell is decreased relative to the SIRT3 protein or RNA level in the non-tumor cell.

### BRIEF DESCRIPTION OF THE DRAWINGS

FIG. 1 shows that the metabolic profiles of SIRT3 KO MEFs reflect an increase in glycolytic pathways and a decrease in mitochondrial oxidative metabolism. (A) Schematic illustrating the metabolites that are increased or decreased in SIRT3 KO MEFs compared to SIRT3 WT MEFs (n=4), p<0.1. Metabolites in parentheses were not measured. PPP, pentose phosphate pathway. The nonoxidative and oxidative arms of the PPP are shown. Levels of glycolytic intermediates (B), TCA cycle intermediates (C), glucose (D), glucose-1-phosphate (E) and ribose-5-phosphate (F). Growth curves of SIRT3 WT and KO MEFs (n=3) cultured in media containing glucose (G) or galactose (H). Error bars, ±SD. (1-N) Glucose uptake and lactate production in SIRT3 WT and KO MEFs (n=6). (I) Relative glucose uptake and (J) lactate production in SIRT3 WT and KO MEFs. (K) Relative glucose uptake and (L) relative lactate production in SIRT3 WT and KO MEFs incubated with or without 100 nM rotenone. (M) Glucose uptake and (N) lactate production in SIRT3 WT and KO MEFs cultured in the presence or absence of 50 μg/ml etomoxir. Cells were treated with drugs for 24 hours before measuring glucose uptake and lactate. All error bars (except growth curves), ±SEM. (*) p<0.05; (**) p<0.01, (***) p<0.001.

FIG. 2 shows that SIRT3 KO mice have elevated glucose uptake and hypoxic signatures in vivo. ¹⁸F-fluorodeoxyglucose (¹⁸F-FDG) uptake in the brown adipose tissue (BAT) of SIRT3 WT and KO mice was measured using positron emission tomography-computed tomography (PET/CT). (A) Representative scans indicating relative levels of uptake from low (black) to high (white). (B) Quantification of BAT ¹⁸F-FDG uptake normalized to body weight (n=6). (C) Gene set enrichment analysis of canonical pathways with the ranked genes list from most up- to most down-regulated in SIRT3 KO BAT. (D) Heat map comparing metabolite patterns of SIRT3 deletion and hypoxia. SIRT3 WT and KO MEFs (n=4) were cultured in 21% O₂ (normoxia, N) or 1% O₂ for 12 hours (hypoxia, H) and metabolites were analyzed by LC-MS. Relative levels of glycolytic intermediates (E) and ribose-5-phosphate (F). (G) Glucose uptake of MEFs cultured under hypoxia for 6 hours. Error bars, ±SEM. (*) p<0.05; (**) p<0.01.

FIG. 3 shows that SIRT3 targets metabolic proteins and activates SDH. In A, Anti-FLAG immuoprecipitates of HEK293T cells transiently expressing vector, FLAG-tagged SIRT3, SIRT4 or SIRT5 were immunoblotted with either a cocktail recognizing two subunits of complex II (SDHA and SDHB, as labeled) and a subunit of complex V, an antibody against SUCLA2, an antibody cocktail recognizing representative subunits of each of the five respiratory complexes (1-V) or an antibody against FLAG epitope. In B, Anti-FLAG immunoprecipitates of cells expressing vector or FLAG-tagged SIRT1-7 were immunoblotted with antibodies against SDHA, OSCP and FLAG. In c and d, acetylated proteins (C) and complex II (D), along with GFP as a negative control, were immunoprecipitated from liver mitochondria isolated from SIRT3 WT or KO mice and probed with antibodies recognizing SDHA, acetyl-lysine (AcK) or SIRT3. In E, complex II immunoprecipitates were incubated with recombinant SIRT3, His-tagged SIRT3 (SIRT3-His) or catalytically inactive SIRT3 (SIRT3-H248Y-His) and NAD or nicotinamide (NAM), a sirtuin inhibitor, as indicated, incubated for 2 hours at 37° C. and immunoblotted for acetyl-lysine (AcK), SDHA and SIRT3. In F, malonate-sensitive SDH activity was measured from SIRT3 WT or KO MEFs and normalized to total protein.

FIG. 4 shows a table of acetylated lysines of SDHA. Peptides identified as having acetylated lysines are shown with the residue number corresponding to the acetylated lysine.

FIG. 5 shows that SIRT3 is required for activation of SDH and complex V activity in vivo. In A and B, SDH (A) and complex V (B) activity was measured from mitochondria isolated from fed or 48 h fasted SIRT3 WT or KO mice and activity was normalized to citrate synthase activity to control for the mass of functional mitochondria.

FIG. 6 shows that SIRT3 regulates HIF1α stability. (A) Immunoblots of nuclear extracts from SIRT3 WT and KO MEFs cultured at 21% O₂. Immunoblots of MEFs (B) or HEK293T cells expressing control shRNA (shNS) or shRNA targeted against SIRT3 (C) cultured at 1% O₂ for the indicated times. (D) HIF1α target genes in SIRT3 WT and KO MEFs after 6 hours of hypoxia were measured by qRT-PCR and shown as a ratio of SIRT3 WT normoxia levels. (E) Immunoblots of HEK293T cells stably overexpressing empty vector or SIRT3 cultured at 1% O₂ for the indicated times. (F) Expression of HIF1α target genes in HEK293T control and SIRT3-overexpressing cells after 6 hours of hypoxia. (G) SIRT3 WT and KO MEFs expressing shNS or shRNA against HIF1α (shHIF1#1,2) were cultured in normoxia or hypoxia (6 hours) and the fold change in Glut1 levels was measured by qRT-PCR. (H) Lactate produced by SIRT3 WT and KO MEFs expressing shNS or shHIF1α after 6 hours of hypoxia expressed as a ratio of SIRT3 WT shNS normoxic controls. Significance was assessed by two-way ANOVA. (I) Expression of Glut1 and Hk2 in the brown adipose tissue (left) and heart (right) of SIRT3 WT and KO mice (n=5-7) was measured by qRT-PCR. β-2-microglobulin or Rps16 were used as endogenous controls for qRT-PCR. Error bars, ±SEM (n=4-6). (*) p<0.05; (**) p<0.01.

FIG. 7 shows that SIRT3 regulates HIF1α stability through ROS. (A) Nuclear extracts from shNS and shSIRT3 HEK293T cells treated with or without 10 μM MG-132 for 1 hour or 1 mM DMOG for 4 hours as indicated were immunoblotted with antibodies specific to hydroxylated HIF1α (HIF—OH) or total HIF1α. (B) Fold induction of HIF1αtarget genes in response to hypoxia (n=6) measured by qRT-PCR. The ratio of hypoxic to normoxic gene expression is shown. (C) Fold induction of Glut1 and Hk2 in response to DMOG treatment was measured by qRT-PCR and the ratio of untreated to DMOG-treated gene expression is shown (n=6). (D) The increase in ROS production with hypoxia was calculated as the fold change in ROS in hypoxic cells relative to normoxic controls. (E) Immunoblots of SIRT3 WT and KO MEFs incubated with 10 mM N-acetylcysteine (NAC) and cultured under hypoxia. (F) Immunoblots of SIRT3 WT and KO MEFs cultured at 21% O₂ with 10 mM NAC or 1 mM DMOG as indicated. (G) Glut1 expression was measured by qRT-PCR in SIRT3 WT and KO MEFs (n=5) that were incubated with 10 mM NAC and cultured under hypoxia. Significance was assessed by one-way ANOVA. (G) Growth curves of SIRT3 WT and KO MEFs (n=3) cultured in standard media or media supplemented with 10 mM NAC. Error bars, ±SD. Protein carbonyls (I) and lipid peroxidation (J) were measured in brown adipose tissue (BAT) of SIRT3 WT and KO mice (n=7). (K) qRT-PCR analysis of Glut1 expression in BAT of SIRT3 WT and KO mice treated with 40 mM NAC. β-2-microglobulin or Rps16 were used as endogenous controls. Error bars (except for growth curves), ±SEM. (*) p<0.05; (**) p<0.01.

FIG. 8 shows that SIRT3 is significantly deleted in human breast cancer. (A) Soft agar assays using transformed SIRT3 WT and KO MEFs expressing shNS or shRNA against HIF1α (shHIF1) (n=4). (B) Quantitative RT-PCR on RNA isolated from xenograft tumors and normalized to expression of 36B4. (C) Hematoxylin and eosin (H&E) staining (left) and immunohistochemial analysis of GLUT1 expression (right) in xenograft tumors. One representative pair of contralateral tumors is shown. Scale bar, 50 μm. (D) Table summarizing SIRT3 deletion frequency across a panel of human tumors. (E) Schematic of copy number changes at the SIRT3-5 and TP53 loci. Amplifications are shown in red; deletions are shown in blue. (F) Expression levels of SIRT3 and several HIF1α target genes were determined using the Oncomine cancer microarray database in normal versus breast cancers. (G) Linear regression of SIRT3 and GLUT1 across the panel of normal and breast cancer samples described in (F). (H) Representative image of SIRT3 expression in normal breast epithelium and in breast tumor cells as assessed by immunohistochemistry. SIRT3 levels were classified as absent (O), weak scattered (1) or positive as strong (2) compared to normal epithelium and the percentage of patients classified in each category is depicted in histogram at right. Error bars, ±SEM (n=4-6). (*) p<0.05; (**) p<0.01.

FIG. 9 shows the effect of loss of SIRT3 in vivo leads to tumorigenesis. In (A) depicts the relative colony formation rate of SIRT3 WT and KO MEFs when cultured in media containing galactose instead of glucose. In B, the percent tumor formation in nude mice injected with SIRT3 WT or KO MEFs transformed with retroviral expression of the Ras and E1a oncogenes is shown. In C, mice were injected with WT MEFs on the right flank and KO MEFs on the left flank and tumors are shown after dissection or in the animal. D and E depict growth curves from two independent xenograft experiments: (D) 5.0×10⁶ cells were injected into each flank of 10 nude mice, (E) 7.5×10⁶ cells were injected into each flank of 11 nude mice. In each experiment, mice were injected with WT cells on one flank and KO cells on the other. Visible tumor volume was measured on the indicated days (n=3-8). In F, the mass of dissected tumors were pooled between two experiments, distinguished by black or grey circles. Each circle represents one mouse, and the line connects tumors from a single mouse. Circles without lines indicate that the particular mouse failed to grow a SIRT3 WT tumor. G and H depict the frequency of deletion of SIRT3 and TP53, respectively, in the indicated cancer types. I depicts the fold reduction in SIRT3 expression in breast cancer tissue samples.

FIG. 10 shows an immunoblot depicting the stable expression of SIRT3 in three independent breast cancer cell lines: MCF7, T47D and CAMA1.

FIG. 11 shows that SIRT3 suppresses the Warburg effect in human breast cancer cells. (A) Lactate production and (B) glucose consumption of MCF7, T47D and CAMA1 cells stably expressing empty vector or SIRT3 and cultured under hypoxia expressed as a ratio of empty-vector normoxic controls. (C) Relative glucose uptake and (D) relative lactate production in CAMA1 control or SIRT3 overexpressing cells incubated with or without 100 nM rotenone. (E) Glucose uptake and (F) lactate production in CAMA1 cell lines cultured in the presence or absence of 50 μg/ml etomoxir. (G) Immunoblots of CAMA1 cells stably expressing control vector or SIRT3-FLAG cultured at 1% oxygen for the indicated. (H) qRT-PCR of HIF1α target genes in CAMA1 cells cultured at 1% oxygen. (I) Induction of HIF1α target genes in response to hypoxia measured by qRT-PCR in CAMA1 cells. The ratio of normoxic to hypoxic gene expression in each cell line is shown. (J) Induction of HIF1α target genes in response to 1 mM DMOG treatment measured by qRT-PCR in CAMA1 cells. The ratio of untreated to DMOG-treated gene expression in each cell line is shown. Growth curves of CAMA1 cells (n=3) cultured in glucose (K) or galactose (L). Error bars, ±SD. (M) Schematic of the regulation of HIF1α and the Warburg effect by SIRT3. β-2-microglobulin was used as an endogenous control for qRT-PCR. Error bars (except for growth curves), ±SEM. (*) p<0.05; (**) p<0.01.

### DETAILED DESCRIPTION OF THE INVENTION

### General

In some embodiments, the present invention relates to methods of preventing or treating cancer and/or decreasing damage to organs or tissues exposed to hypoxic conditions through the use of agents that modulate the expression or activity of SIRT3.

As described herein, SIRT3 redirects cellular metabolism, acting as a gatekeeper to control flux through the citric acid cycle (the tricarboxylic acid cycle, or the TCA cycle). It is likely that SIRT3 regulates multiple steps of OXPHOS and the TCA cycle. By modulating the activity of multiple mitochondrial enzymes, SIRT3 controls flux through mitochondrial pathways to promote a systematic metabolic shift toward oxidative metabolism. In this way, SIRT3 reversibly regulates mitochondrial enzymes to control global fuel utilization, and the loss of metabolic plasticity in SIRT3 null cells results in unregulated glycolysis, cell growth and tumor progression.

The switch from oxidative to glycolytic metabolism often occurs during tumorigenesis. This reprogramming of cell metabolism towards a highly glycolytic phenotype, known as the Warburg effect, enables cancer cells to survive and proliferate in the hypoxic environment that is present in many solid tumors. Thus, in certain embodiments, the instant invention relates to methods of increasing the activation or expression of SIRT3 in order to reduce the level of glycolysis in cancer cells. Such methods are useful, for example, in the prevention and treatment of tumors that have undergone metabolic reprogramming.

Organs and tissues are often exposed to hypoxic environments during situations when they experience impaired access to the blood supply, such as occurs during a stroke, myocardial infarction or peripheral vascular disease. In such situations, the damage to the tissue or organ can be reduced by inducing glycolysis in the blood-deprived cells. Thus, in certain embodiments, the instant invention relates to methods of inhibiting the activation or expression of SIRT3 in order to increase the level of glycolysis in cells that are exposed to hypoxic environments. Such methods are useful, for example, in the reduction of damage to organs or tissues exposed to hypoxic conditions following stroke, myocardial infarction or peripheral vascular disease.

### DEFINITIONS

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering.

The terms “agent” are used herein to denote a chemical compound, a small molecule, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. Agents may be identified as having a particular activity by screening assays described herein below. The activity of such agents may render them suitable as a “therapeutic agent” which is a biologically, physiologically, or pharmacologically active substance (or substances) that acts locally or systemically in a subject.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

“Biologically active portion of SIRT3” refers to a portion of SIRT3 protein having a biological activity, such as the ability to deacetylate. Biologically active portions of a SIRT3 may comprise the core domain of SIRT3.

As used herein, the term “cancer” includes, but is not limited to, solid tumors and blood borne tumors. The term cancer includes diseases of the skin, tissues, organs, bone, cartilage, blood and vessels. The term “cancer” further encompasses primary and metastatic cancers.

As used herein, the term “glycolytic phenotype” or “highly glycolytic phenotype” refers to a cell or tumor that is preferentially undergoing glycolytic metabolism rather than oxidative metabolism. Such cells may, for example, have reduced levels of intracellular glucose and elevated levels of glucose-1-phosphate. Such cells may have, for example, elevated levels of one or more intermediates of glycolysis (e.g., F16DP, F26DP, G16DP, DHAP, 3-phosphoglycerate, 2-phosphoglycerate, PEP) and/or reduced levels of TCA metabolites (e.g., pyruvate, citrate, aconitate, succinate). Highly glycolytic cells and tumors can be detected, for example, by monitoring the uptake of 2-¹⁸F-2-deoxyglucose (FDG) (a radioactive modified hexokinase substrate) using positron emission tomography.

The term “isolated polypeptide” refers to a polypeptide, in certain embodiments prepared from recombinant DNA or RNA, or of synthetic origin, or some combination thereof, which (1) is not associated with proteins that it is normally found with in nature, (2) is isolated from the cell in which it normally occurs, (3) is isolated free of other proteins from the same cellular source, (4) is expressed by a cell from a different species, or (5) does not occur in nature.

The term “isolated nucleic acid” refers to a polynucleotide of genomic, cDNA, or synthetic origin or some combination there of, which (1) is not associated with the cell in which the “isolated nucleic acid” is found in nature, or (2) is operably linked to a polynucleotide to which it is not linked in nature.

The term “modulation”, when used in reference to a functional property or biological activity or process (e.g., enzyme activity or receptor binding), refers to the capacity to either up regulate (e.g., activate or stimulate), down regulate (e.g., inhibit or suppress) or otherwise change a quality of such property, activity or process. In certain instances, such regulation may be contingent on the occurrence of a specific event, such as activation of a signal transduction pathway, and/or may be manifest only in particular cell types.

A “modulator” may be a polypeptide, nucleic acid, macromolecule, complex, molecule, small molecule, compound, species or the like (naturally-occurring or non-naturally-occurring), or an extract made from biological materials such as bacteria, plants, fungi, or animal cells or tissues, that may be capable of causing modulation. Modulators may be evaluated for potential activity as inhibitors or activators (directly or indirectly) of a functional property, biological activity or process, or combination of them, (e.g., agonist, partial antagonist, partial agonist, inverse agonist, antagonist, anti-microbial agents, inhibitors of microbial infection or proliferation, and the like) by inclusion in assays. In such assays, many modulators may be screened at one time. The activity of a modulator may be known, unknown or partially known.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a non-natural arrangement.

A “patient” or “subject” refers to either a human or a non-human animal.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” is art-recognized and refers to the relatively non-toxic, inorganic and organic acid addition salts of compounds, including, for example, those contained in compositions described herein.

The terms “polypeptide fragment” or “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions may occur at the amino-terminus or carboxy-terminus of the reference polypeptide, or alternatively both. Fragments typically are at least 5, 6, 8 or 10 amino acids long, at least 14 amino acids long, at least 20, 30, 40 or 50 amino acids long, at least 75 amino acids long, or at least 100, 150, 200, 300, 500 or more amino acids long. A fragment can retain one or more of the biological activities of the reference polypeptide. In certain embodiments, a fragment may comprise a druggable region, and optionally additional amino acids on one or both sides of the druggable region, which additional amino acids may number from 5, 10, 15, 20, 30, 40, 50, or up to 100 or more residues. Further, fragments can include a sub-fragment of a specific region, which sub-fragment retains a function of the region from which it is derived. In another embodiment, a fragment may have immunogenic properties. Fragments may be devoid of about 1, 2, 5, 10, 20, 50, 100 or more amino acids at the N- or C-terminus of the wildtype protein.

The term “SIRT3-activating compound” or “agent that increases SIRT3 activity” refers to an agent that increases the level of SIRT3 protein and/or increases at least one activity of a SIRT3 protein. In an exemplary embodiment, a SIRT3-activating compound may increase at least one biological activity of a SIRT3 protein by at least about 10%, 25%, 50%, 75%, 100%, or more. Exemplary biological activities of SIRT3 proteins include deacetylation, destabilization of HIF1α and inhibition of glycolysis.

The term “SIRT3-inhibiting compound” or “agent that decreases SIRT3 activity” refers to an agent that decreases the level of SIRT3 protein and/or decreases at least one activity of a SIRT3 protein. In an exemplary embodiment, a SIRT3-inhibing compound may decrease at least one biological activity of a SIRT3 protein by at least about 10%, 25%, 50%, 75%, 100%, or more. Exemplary biological activities of SIRT3 proteins include deacetylation, destabilization of HIF1α and inhibition of glycolysis.

The term “small molecule” is art-recognized and refers to a composition which has a molecular weight of less than about 2000 amu, or less than about 1000 amu, and even less than about 500 amu. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays described herein. The term “small organic molecule” refers to a small molecule that is often identified as being an organic or medicinal compound, and does not include molecules that are exclusively nucleic acids, peptides or polypeptides.

The phrases “therapeutically-effective amount” and “effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

### SIRT3 Polypeptides

As used herein, the term “SIRT3” or “SIRT3 protein” refers to proteins, e.g., eukaryotic proteins, e.g., mammalian proteins, comprising a mitochondrial matrix protein having deacetylase activity, as well as functional domains, fragments (e.g., functional fragments), e.g., fragments of at least 8 amino acids, e.g., at least 8, 18, 28, 64, 128, 150, 180, 200, 220, 240, 260, or 280 amino acids, and variants thereof. Exemplary functional fragments of SIRT3 can, for example, have deacetylase activity and/or the ability to interact with a Sirt3 binding partner. Exemplary SIRT3 proteins include those designated GenBank NM_{—}001017524 (human SIRT3) and NM_{—}022433 (mouse SIRT3). Homologs of SIRT3 proteins will share 60%, 80%, 85%, 90%, 95%, 98%, 99% sequence identity to a known SIRT3 protein and, e.g., feature deacetylase activity. Variants of SIRT3 proteins can be produced by standard means, including site-directed and random mutagenesis.

In certain embodiments, a protein described herein is further linked to a heterologous polypeptide, e.g., a polypeptide comprising a domain which increases its solubility and/or facilitates its purification, identification, detection, and/or structural characterization. A protein described herein may be linked to at least 2, 3, 4, 5, or more heterologous polypeptides. Polypeptides may be linked to multiple copies of the same heterologous polypeptide or may be linked to two or more heterologous polypeptides. The fusions may occur at the N-terminus of the polypeptide, at the C-terminus of the polypeptide, or at both the N- and C-terminus of the polypeptide. It is also within the scope of the invention to include linker sequences between a protein described herein and the fusion domain in order to facilitate construction of the fusion protein or to optimize protein expression or structural constraints of the fusion protein.

In another embodiment, a protein may be modified so that its rate of traversing the cellular membrane is increased. For example, the polypeptide may be fused to a second peptide which promotes “transcytosis,” e.g., uptake of the peptide by cells. The peptide may be a portion of the HIV transactivator (TAT) protein, such as the fragment corresponding to residues 37-62 or 48-60 of TAT, portions which have been observed to be rapidly taken up by a cell in vitro (Green and Loewenstein, (1989) Cell 55:1179-1188). Alternatively, the internalizing peptide may be derived from the Drosophila antennapedia protein, or homologs thereof. The 60 amino acid long homeodomain of the homeo-protein antennapedia has been demonstrated to translocate through biological membranes and can facilitate the translocation of heterologous polypeptides to which it is coupled. Thus, the polypeptide may be fused to a peptide consisting of about amino acids 42-58 of Drosophila antennapedia or shorter fragments for transcytosis (Derossi et al. (1996) J Biol Chem 271:18188-18193; Derossi et al. (1994) J Biol Chem 269:10444-10450; and Perez et al. (1992) J Cell Sci 102:717-722). The transcytosis polypeptide may also be a non-naturally-occurring membrane-translocating sequence (MTS), such as the peptide sequences disclosed in U.S. Pat. No. 6,248,558.

In certain embodiments, it may be advantageous to provide naturally-occurring or experimentally-derived homologs of the polypeptide of the invention. Such homologs may function as a modulator to promote or inhibit a subset of the biological activities of the naturally-occurring form of the polypeptide. Thus, specific biological effects may be elicited by treatment with a homolog of limited function, and with fewer side effects relative to treatment with agonists or antagonists which are directed to all of the biological activities of the polypeptide of the invention. For instance, antagonistic homologs may be generated which interfere with the ability of the wild-type polypeptide of the invention to associate with certain proteins, but which do not substantially interfere with the formation of complexes between the native polypeptide and other cellular proteins.

### SIRT3 Nucleic Acids

Nucleic acids encoding any of the polypeptides described herein are also provided herein. A nucleic acid may further be linked to a promoter and/or other regulatory sequences, as further described herein. Exemplary nucleic acids are those that are at least about 80%, 85%, 90%, 95%, 98%, 99% or 100% identical to a nucleotide sequence provided herein or a fragment thereof, such as nucleic acid sequence encoding the protein fragments described herein. Nucleic acids may also hybridize specifically, e.g., under stringent hybridization conditions, to a nucleic acid described herein or a fragment thereof.

Nucleic acids, e.g., those encoding a protein of interest or functional homolog thereof, or a nucleic acid intended to inhibit the production of a protein of interest (e.g., siRNA, shRNA or antisense RNA) can be delivered to cells in culture, ex vivo, and in vivo. The cells can be of any type including without limitation cancer cells, stem cells, neuronal cells, myocytes, and non-neuronal cells. The delivery of nucleic acids can be by any technique known in the art including viral mediated gene transfer, liposome mediated gene transfer, direct injection into a target tissue, organ, or tumor, injection into vasculature which supplies a target tissue or organ.

Polynucleotides can be administered in any suitable formulations known in the art. These can be as virus particles, as naked DNA, in liposomes, in complexes with polymeric carriers, etc. Polynucleotides can be administered to the arteries which feed a tissue or tumor. They can also be administered to adjacent tissue, whether tumor or normal, which could express the demethylase protein.

Nucleic acids can be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus). Nucleic acids can be administered in any desired format that provides sufficiently efficient delivery levels, including in virus particles, in liposomes, in nanoparticles, and complexed to polymers.

A polynucleotide of interest can also be combined with a condensing agent to form a gene delivery vehicle. The condensing agent may be a polycation, such as polylysine, polyarginine, polyornithine, protamine, spermine, spermidine, and putrescine. Many suitable methods for making such linkages are known in the art.

In an alternative embodiment, a polynucleotide of interest is associated with a liposome to form a gene delivery vehicle. Liposomes are small, lipid vesicles comprised of an aqueous compartment enclosed by a lipid bilayer, typically spherical or slightly elongated structures several hundred Angstroms in diameter. Under appropriate conditions, a liposome can fuse with the plasma membrane of a cell or with the membrane of an endocytic vesicle within a cell which has internalized the liposome, thereby releasing its contents into the cytoplasm. Prior to interaction with the surface of a cell, however, the liposome membrane acts as a relatively impermeable barrier which sequesters and protects its contents, for example, from degradative enzymes. Additionally, because a liposome is a synthetic structure, specially designed liposomes can be produced which incorporate desirable features. See Stryer, Biochemistry, pp. 236-240, 1975 (W. H. Freeman, San Francisco, Calif.); Szoka et al., Biochim. Biophys. Acta 600:1, 1980; Bayer et al., Biochim. Biophys. Acta. 550:464, 1979; Rivnay et al., Meth. Enzymol. 149:119, 1987; Wang et al., PROC. NATL. ACAD. SCI. U.S.A. 84: 7851, 1987, Plant et al., Anal. Biochem. 176:420, 1989, and U.S. Pat. No. 4,762,915. Liposomes can encapsulate a variety of nucleic acid molecules including DNA, RNA, plasmids, and expression constructs comprising growth factor polynucleotides such those disclosed in the present invention.

Liposomal preparations for use in the present invention include cationic (positively charged), anionic (negatively charged) and neutral preparations. Cationic liposomes have been shown to mediate intracellular delivery of plasmid DNA (Felgner et al., Proc. Natl. Acad. Sci. USA 84:7413-7416, 1987), mRNA (Malone et al., Proc. Natl. Acad. Sci. USA 86:6077-6081, 1989), and purified transcription factors (Debs et al., J. Biol. Chem. 265:10189-10192, 1990), in functional form. Cationic liposomes are readily available. For example, N[1-2,3-dioleyloxy)propyl]-N,N,N-triethylammonium (DOTMA) liposomes are available under the trademark Lipofectin, from GIBCO BRL, Grand Island, N.Y. See also Felgner et al., Proc. Natl. Acad. Sci. USA 91: 5148-5152.87, 1994. Other commercially available liposomes include Transfectace (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA 75:4194-4198, 1978; and WO 90/11092 for descriptions of the synthesis of DOTAP (1,2-bis(oleoyloxy)-3-(trimethylammonio)propane) liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

One or more polypeptide (e.g., a SIRT3 protein, or a polypeptide that modulates SIRT3 activity) or nucleic acid (e.g., siRNA) of interest may be encoded by a single nucleic acid. Alternatively, separate nucleic acids may encode different protein or nucleic acids of interest. Different species of nucleic acids may be in different forms; they may use different promoters or different vectors or different delivery vehicles. Similarly, the same protein or nucleic acid of interest may be used in a combination of different forms.

### Inhibitory RNA Molecules

In certain embodiments, inhibitory RNA molecules that specifically target SIRT3 mRNA (e.g., antisense molecules, siRNA or shRNA molecules, ribozymes or triplex molecules) are used in methods of the invention. Such molecules are useful, for example, in methods of increasing glycolysis in a cell, methods of increasing cell survival under hypoxic conditions, methods of treating or preventing damage to a tissue or organ that has been exposed to hypoxic conditions and methods of reducing the damage caused by a stroke, a myocardial infarction or a peripheral vascular disease.

The inhibitory RNA molecules of the invention may be contacted with a cell or administered to an organism. Alternatively, constructs encoding these may be contacted with or introduced into a cell or organism. Antisense constructs, antisense oligonucleotides, RNA interference constructs or siRNA duplex RNA molecules can be used to interfere with expression of a protein of interest, e.g., a SIRT3 protein. Typically at least 15, 17, 19, or 21 nucleotides of the complement of the SIRT3 mRNA sequence are sufficient for an antisense molecule. Typically at least 19, 21, 22, or 23 nucleotides of a target sequence are sufficient for an RNA interference molecule. The RNA interference molecule may have a 2 nucleotide 3′ overhang. If the RNA interference molecule is expressed in a cell from a construct, for example from a hairpin molecule or from an inverted repeat of the desired histone demethylase sequence, then the endogenous cellular machinery will create the overhangs. Inhibitory RNA molecules can be prepared by chemical synthesis, in vitro transcription, or digestion of long dsRNA by Rnase III or Dicer. These can be introduced into cells by transfection, electroporation, or other methods known in the art. See Hannon, G J, 2002, RNA Interference, Nature 418: 244-251; Bernstein E et al., 2002, The rest is silence. RNA 7: 1509-1521; Hutvagner G et al., RNAi: Nature abhors a double-strand. Curr. Opin. Genetics & Development 12: 225-232; Brummelkamp, 2002, A system for stable expression of short interfering RNAs in mammalian cells. Science 296: 550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052.

Antisense or RNA interference molecules can be delivered in vitro to cells or in vivo, e.g., to tumors or hypoxic tissues of a mammal. Typical delivery means known in the art can be used. For example, delivery to a tumor can be accomplished by intratumoral injections. Other modes of delivery can be used without limitation, including: intravenous, intramuscular, intraperitoneal, intraarterial, local delivery during surgery, endoscopic, subcutaneous, and per os. In a mouse model, the antisense or RNA interference can be adminstered to a tumor cell in vitro, and the tumor cell can be subsequently administered to a mouse. Vectors can be selected for desirable properties for any particular application. Vectors can be viral or plasmid. Adenoviral vectors are useful in this regard. Tissue-specific, cell-type specific, or otherwise regulatable promoters can be used to control the transcription of the inhibitory polynucleotide molecules. Non-viral carriers such as liposomes or nanospheres can also be used.

### Modulators of SIRT3

Certain embodiments of the present invention relate to methods of preventing or treating cancer or of reducing damage to organs or tissues exposed to hypoxic conditions. These methods involve administering an agent that either increases or decreases the activity and/or expression of SIRT3. Agents which may be used to modulate the activity of SIRT3 include antibodies (e.g., conjugated antibodies), proteins, peptides, small molecules and inhibitory RNA molecules, e.g., siRNA molecules, shRNA, ribozymes, and antisense oligonucleotides.

Any agent that modulates SIRT3 can be used to practice certain methods of the invention. Such agents can be those described herein, those known in the art, or those identified through routine screening assays (e.g. the screening assays described herein).

In certain embodiments, the agent increases the activity or expression of SIRT3. Such molecules are useful, for example, in methods of treating cancer, including solid tumors and cancers with a highly glycolyic phenotype. SIRT3-activating agents can include, for example, SIRT3 proteins or polypeptides, SIRT3 nucleic acids, and small molecule activators of SIRT3. Examples of small molecule activators of SIRT3 include those described in published U.S. patent applications US2009/0163476, US2009/0012080, US2009/0105246, and US2006/0229265 each of which is hereby incorporated by reference in its entirety.

In certain embodiments, the agent decreases the activity or expression of SIRT3. Such molecules are useful, for example, in methods of reducing damage to organs or tissues exposed to hypoxic conditions such as occurs during stroke, myocardial infarction and peripheral vascular disease. SIRT3-inhibiting agents can include, for example, homologs of SIRT3 proteins or polypeptides that lack deacetylase activity, inhibitory RNA molecules, and small molecule inhibits of SIRT3. Examples of small molecule activators of SIRT3 include those described in published U.S. patent application US2008/0287653, which is hereby incorporated by reference in its entirety.

In some embodiments, assays used to identify agents useful in the methods of the present invention include a reaction between SIRT3 and one or more assay components. The other components may be either a test compound (e.g. the potential agent), or a combination of test compounds and a natural binding partner or deacetylation target of SIRT3 (e.g. acetyl-CoA synthetase 2). Agents identified via such assays, such as those described herein, may be useful, for example, for preventing or treating cancer or of reducing damage to organs or tissues exposed to hypoxic conditions.

Agents useful in the methods of the present invention may be obtained from any available source, including systematic libraries of natural and/or synthetic compounds. Agents may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of agents may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

Agents useful in the methods of the present invention may be identified, for example, using assays for screening candidate or test compounds which modulate the activity of SIRT3 or a biologically active portion thereof on SIRT3 substrates (e.g., acetyl-CoA synthetase 2). For example, candidate or test compounds can be screened for the ability to modulate the protein deacetylase activity of SIRT3.

The basic principle of the assay systems used to identify compounds that modulate the activity of SIRT3 involves preparing a reaction mixture containing SIRT3 and its substrate under conditions and for a time sufficient to allow SIRT3 to deacetylate the substrate. In order to test an agent for modulatory activity, the reaction mixture is prepared in the presence and absence of the test compound. The test compound can be initially included in the reaction mixture, or can be added at a time subsequent to the addition of SIRT3 and its binding partner. Control reaction mixtures are incubated without the test compound or with a placebo. The deacetylation of the substrate by SIRT3 is then detected. Deacetylation can be detected by any method known in the art including, but not limited to, using anti-acetylated lysine antibodies or radioactively labeled acetyl groups to detect the level of substrate acetylation. The deacetylation of the substrate in the control reaction, but less or no such deacetylation in the reaction mixture containing the test compound, indicates that the compound decreases with the activity of SIRT3. Conversely, elevated levels of deacetylation in the presence of the compound compared to the control reaction indicate that the compound may increases the deacetylation activity of SIRT3.

In another embodiment, agents useful in the methods of the invention may be identified using assays for screening candidate or test compounds which bind to SIRT3 or a biologically active portion thereof. Determining the ability of the test compound to directly bind to SIRT3 can be accomplished, for example, by coupling the compound with a radioisotope or enzymatic label such that binding of the compound to SIRT3 can be determined by detecting the labeled compound in a complex. For example, compounds can be labeled with ¹²⁵I, ³⁵S, ¹⁴C, or ³H, either directly or indirectly, and the radioisotope detected by direct counting of radioemission or by scintillation counting. Alternatively, assay components can be enzymatically labeled with, for example, horseradish peroxidase, alkaline phosphatase, or luciferase, and the enzymatic label detected by determination of conversion of an appropriate substrate to product.

Agents useful in the methods of the invention may also be identified, for example, using assays that identify compounds which modulate (e.g., affect either positively or negatively) interactions between SIRT3 and a substrate and/or binding partners (e.g. acetyl-CoA synthetase 2).

The basic principle of the assay systems used to identify compounds that modulate the interaction between SIRT3 and its binding partner involves preparing a reaction mixture containing SIRT3 and its binding partner under conditions and for a time sufficient to allow the two products to interact and bind, thus forming a complex. In order to test an agent for inhibitory activity, the reaction mixture is prepared in the presence and absence of the test compound. The test compound can be initially included in the reaction mixture, or can be added at a time subsequent to the addition of SIRT3 and its binding partner. Control reaction mixtures are incubated without the test compound or with a placebo. The formation of any complexes between SIRT3 and its binding partner is then detected. The formation of a complex in the control reaction, but less or no such formation in the reaction mixture containing the test compound, indicates that the compound interferes with the interaction of SIRT3 and its binding partner. Conversely, the formation of more complex in the presence of the compound than in the control reaction indicates that the compound may enhance interaction of SIRT3 and its binding partner.

The assay for compounds that modulate the interaction of SIRT3 with its binding partner may be conducted in a heterogeneous or homogeneous format. Heterogeneous assays involve anchoring either SIRT3 or its binding partner onto a solid phase and detecting complexes anchored to the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between SIRT3 and the binding partner (e.g., by competition) can be identified by conducting the reaction in the presence of the test substance, i.e., by adding the test substance to the reaction mixture prior to or simultaneously with SIRT3 and its interactive binding partner. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either SIRT3 or its binding partner is anchored onto a solid surface or matrix, while the other corresponding non-anchored component may be labeled, either directly or indirectly. In practice, microtitre plates are often utilized for this approach. The anchored species can be immobilized by a number of methods, either non-covalent or covalent, that are typically well known to one who practices the art. Non-covalent attachment can often be accomplished simply by coating the solid surface with a solution of SIRT3 or its binding partner and drying. Alternatively, an immobilized antibody specific for the assay component to be anchored can be used for this purpose.

In related assays, a fusion protein can be provided which adds a domain that allows one or both of the assay components to be anchored to a matrix. For example, glutathione-S-transferase/marker fusion proteins or glutathione-S-transferase/binding partner can be adsorbed onto glutathione sepharose beads (Sigma Chemical, St. Louis, Mo.) or glutathione derivatized microtiter plates, which are then combined with the test compound or the test compound and either the non-adsorbed SIRT3 or its binding partner, and the mixture incubated under conditions conducive to complex formation (e.g., physiological conditions). Following incubation, the beads or microtiter plate wells are washed to remove any unbound assay components, the immobilized complex assessed either directly or indirectly, for example, as described above. Alternatively, the complexes can be dissociated from the matrix, and the level of SIRT3 binding or activity determined using standard techniques.

A homogeneous assay may also be used to identify modulators of SIRT3. This is typically a reaction, analogous to those mentioned above, which is conducted in a liquid phase in the presence or absence of the test compound. The formed complexes are then separated from unreacted components, and the amount of complex formed is determined. As mentioned for heterogeneous assay systems, the order of addition of reactants to the liquid phase can yield information about which test compounds modulate (inhibit or enhance) complex formation and which disrupt preformed complexes.

In such a homogeneous assay, the reaction products may be separated from unreacted assay components by any of a number of standard techniques, including but not limited to: differential centrifugation, chromatography, electrophoresis and immunoprecipitation. In differential centrifugation, complexes of molecules may be separated from uncomplexed molecules through a series of centrifugal steps, due to the different sedimentation equilibria of complexes based on their different sizes and densities (see, for example, Rivas, G., and Minton, A. P., Trends Biochem Sci 1993 August; 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the complex as compared to the uncomplexed molecules may be exploited to differentially separate the complex from the remaining individual reactants, for example through the use of ion-exchange chromatography resins. Such resins and chromatographic techniques are well known to one skilled in the art (see, e.g., Heegaard, 1998, J Mol. Recognit. 11:141-148; Hage and Tweed, 1997, J. Chromatogr. B. Biomed. Sci. Appl., 699:499-525). Gel electrophoresis may also be employed to separate complexed molecules from unbound species (see, e.g., Ausubel et al (eds.), In: Current Protocols in Molecular Biology, J. Wiley & Sons, New York. 1999). In this technique, protein or nucleic acid complexes are separated based on size or charge, for example. In order to maintain the binding interaction during the electrophoretic process, nondenaturing gels in the absence of reducing agent are typically preferred, but conditions appropriate to the particular interactants will be well known to one skilled in the art. Immunoprecipitation is another common technique utilized for the isolation of a protein-protein complex from solution (see, e.g., Ausubel et at (eds.), In: Current Protocols in Molecular Biology, J. Wiley & Sons, New York. 1999). In this technique, all proteins binding to an antibody specific to one of the binding molecules are precipitated from solution by conjugating the antibody to a polymer bead that may be readily collected by centrifugation. The bound assay components are released from the beads (through a specific proteolysis event or other technique well known in the art which will not disturb the protein-protein interaction in the complex), and a second immunoprecipitation step is performed, this time utilizing antibodies specific for the correspondingly different interacting assay component. In this manner, only formed complexes should remain attached to the beads. Variations in complex formation in both the presence and the absence of a test compound can be compared, thus offering information about the ability of the compound to modulate interactions between SIRT3 and its binding partner.

Modulators of SIRT3 expression may also be identified, for example, using methods wherein a cell is contacted with a candidate compound and the expression of SIRT3 mRNA or protein is determined. The level of expression of mRNA or protein in the presence of the candidate compound is compared to the level of expression of mRNA or protein in the absence of the candidate compound. The candidate compound can then be identified as a modulator of SIRT3 expression based on this comparison. For example, when expression of SIRT3 is greater in the presence of the candidate compound than in its absence, the candidate compound is identified as a stimulator of SIRT3 mRNA or protein expression. Conversely, when expression of SIRT3 is less in the presence of the candidate compound than in its absence, the candidate compound is identified as an inhibitor of SIRT3 mRNA or protein expression.

### Pharmaceutical Compositions

Pharmaceutical compositions of the invention include any modulator of SIRT3 activity or expression (e.g., any small molecule, protein, polypeptide, polynucleotide, or inhibitory RNA molecule that either inhibits or activates the activity or expression of SIRT3), or a pharmaceutically acceptable salt thereof, and a pharmaceutically acceptable carrier or vehicle. Pharmaceutical compositions of the invention that include agents that increase SIRT3 activity or expression are useful for treating cancers (e.g., solid tumors). Pharmaceutical compositions of the invention that include agents that decrease SIRT3 activity or expression are useful for reducing damage to tissues or organs under hypoxic conditions (e.g., during a stroke, myocardial infarction or peripheral vascular disease).

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the compound which produces a therapeutic effect.

In certain embodiments, the pharmaceutical compositions of the invention are useful for the prevention or treatment of cancer. Such compositions may comprise one or more agents that increase SIRT3 activity and/or expression and a second chemotherapeutic agent.

The term chemotherapeutic agent includes, without limitation, platinum-based agents, such as carboplatin and cisplatin; nitrogen mustard alkylating agents; nitrosourea alkylating agents, such as carmustine (BCNU) and other alkylating agents; antimetabolites, such as methotrexate; purine analog antimetabolites; pyrimidine analog antimetabolites, such as fluorouracil (5-FU) and gemcitabine; hormonal antineoplastics, such as goserelin, leuprolide, and tamoxifen; natural antineoplastics, such as taxanes (e.g., docetaxel and paclitaxel), aldesleukin, interleukin-2, etoposide (VP-16), interferon alfa, and tretinoin (ATRA); antibiotic natural antineoplastics, such as bleomycin, dactinomycin, daunorubicin, doxorubicin, and mitomycin; and vinca alkaloid natural antineoplastics, such as vinblastine and vincristine.

Further, the following drugs may also be used in combination with an antineoplastic agent, even if not considered antineoplastic agents themselves: dactinomycin; daunorubicin HCl; docetaxel; doxorubicin HCl; epoetin alfa; etoposide (VP-16); ganciclovir sodium; gentamicin sulfate; interferon alfa; leuprolide acetate; meperidine HCl; methadone HCl; ranitidine HCl; vinblastin sulfate; and zidovudine (AZT). For example, fluorouracil has recently been formulated in conjunction with epinephrine and bovine collagen to form a particularly effective combination.

Still further, the following listing of amino acids, peptides, polypeptides, proteins, polysaccharides, and other large molecules may also be used: interleukins 1 through 18, including mutants and analogues; interferons or cytokines, such as interferons α, β, and γ; hormones, such as luteinizing hormone releasing hormone (LHRH) and analogues and, gonadotropin releasing hormone (GnRH); growth factors, such as transforming growth factor-β (TGF-β), fibroblast growth factor (FGF), nerve growth factor (NGF), growth hormone releasing factor (GHRF), epidermal growth factor (EGF), fibroblast growth factor homologous factor (FGFHF), hepatocyte growth factor (HGF), and insulin growth factor (IGF); tumor necrosis factor-α & β (TNF-α & β); invasion inhibiting factor-2 (IIF-2); bone morphogenetic proteins 1-7 (BMP 1-7); somatostatin; thymosin-α-1; γ-globulin; superoxide dismutase (SOD); complement factors; anti-angiogenesis factors; antigenic materials; and pro-drugs.

Chemotherapeutic agents for use with the compositions and methods of treatment described herein include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegal1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; and pharmaceutically acceptable salts, acids or derivatives of any of the above.

In another embodiment, the composition of the invention may comprise other biologically active substances, including therapeutic drugs or pro-drugs, for example, other chemotherapeutic agents, scavenger compounds, antibiotics, anti-virals, anti-fungals, anti-inflammatories, vasoconstrictors and anticoagulants, antigens useful for cancer vaccine applications or corresponding pro-drugs.

Exemplary scavenger compounds include, but are not limited to thiol-containing compounds such as glutathione, thiourea, and cysteine; alcohols such as mannitol, substituted phenols; quinones, substituted phenols, aryl amines and nitro compounds.

Various forms of the chemotherapeutic agents and/or other biologically active agents may be used. These include, without limitation, such forms as uncharged molecules, molecular complexes, salts, ethers, esters, amides, and the like, which are biologically active.

In certain embodiments, the pharmaceutical compositions of the invention are useful for the reduction of damage to tissues or organs exposed to a hypoxia. Tissues and organs are often exposed to hypoxic conditions during a stroke, a myocardial infarction or a peripheral vascular disease. Such compositions may comprise one or more agents that decrease SIRT3 activity and/or expression and a second therapeutic agent. Examples of therapeutic agents that can be combined with agents that decreases SIRT3 activity and/or expression include, for example, alteplase, aspirin, clopidogrel, dipyridamole, morphine, nitroglycerin, statins and tissue plasminogen activator.

### Exemplary Methods of Treatment, Prevention and Diagnosis of Disease

Provided herein are methods of treatment, prevention and/or diagnosis of conditions and diseases that can be improved by modulating the activity of SIRT3. In some embodiments, the present invention provides therapeutic methods of treating cancer, including a cancerous tumor (e.g., a solid tumor) comprising administering to a subject, (e.g., a subject in need thereof), an effective amount of an agent that increases the expression or activity of SIRT3. In some embodiments the present invention provides therapeutic methods of reducing the damage in a tissue or organ under hypoxic conditions, including during or following a stroke, myocardial infarction or peripheral vascular disease, comprising administering to a subject, (e.g., a subject in need thereof), an effective amount of an agent that decreases the expression or activity of SIRT3. In some embodiments, the present invention provides methods of diagnosing a tumor phenotype (e.g., a metabolic phenotype) comprising determining the level of SIRT3 protein or RNA expressed by the tumor.

The pharmaceutical compositions of the present invention may be delivered by any suitable route of administration, including orally, nasally, as by, for example, a spray, rectally, intravaginally, parenterally, intracisternally and topically, as by powders, ointments or drops, including buccally and sublingually. In certain embodiments the pharmaceutical compositions are delivered generally (e.g., via oral or parenteral administration). In certain other embodiments the pharmaceutical compositions are delivered locally through direct injection into a hypoxic tissue or tumor or direct injection into the hypoxic tissue or tumor's blood supply (e.g., arterial or venous blood supply).

In certain embodiments, the methods of treatment of the present invention comprise administering an agent that increases the activity or expression of SIRT3 in conjunction with a second therapeutic agent to the subject. When used for treating cancer, such methods may comprise administering pharmaceutical compositions comprising an agent that increases the activity or expression of SIRT3 in conjunction with one or more chemotherapeutic agents and/or scavenger compounds, including chemotherapeutic agents described herein, as well as other agents known in the art. When used to reduce damage to a tissue or organ under hypoxic conditions, such methods may comprise administering pharmaceutical compositions comprising an agent that decreases the activity or expression of SIRT3 in conjunction with one or more agents useful for the treatment of stroke, myocardial infarction and/or peripheral vascular disease described herein, as well as other agents known in the art.

Conjunctive therapy includes sequential, simultaneous and separate, or co-administration of the active compound in a way that the therapeutic effects of the first agent administered have not entirely disappeared when the subsequent agent is administered. In certain embodiments, the second agent may be co-formulated with the first agent or be formulated in a separate pharmaceutical composition.

In some embodiments, the subject pharmaceutical compositions of the present invention will incorporate the substance or substances to be delivered in an amount sufficient to deliver to a patient a therapeutically effective amount of an incorporated therapeutic agent or other material as part of a prophylactic or therapeutic treatment. The desired concentration of the active compound in the particle will depend on absorption, inactivation, and excretion rates of the drug as well as the delivery rate of the compound. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

In certain embodiments, the present invention provides therapeutic methods of treating, preventing or diagnosing cancer or a cancer phenotype, including a cancerous tumor (e.g., a solid tumor) in a subject in need thereof. A subject in need thereof may include, for example, a subject who has been diagnosed with a tumor, including a pre-cancerous tumor, a cancer, or a subject who has been treated, including subjects that have been refractory to the previous treatment.

In some embodiments, the methods of the instant invention are used to determine if the tumor has a glycolytic phenotype. As described herein, reduced expression of SIRT3 protein and/or RNA is indicative of a tumor having a glycolytic phenotype. The level of SIRT3 protein and/or RNA can be determined using any method known in the art. For example, in certain embodiments the level of SIRT3 protein is determined using antibodies or antigen binding fragments thereof that bind specifically to SIRT3. In some embodiments the antibodies are either directly or indirectly labeled with a detectable moiety. In some embodiments the SIRT3 RNA level is determined using a nucleic acid amplification assay, such as, for example, a PCR, SDA, TMA or NASBA based assay. In some embodiments the SIRT3 RNA level is determined using a nucleic acid probe that binds specifically to SIRT3 RNA or an amplification product produced from SIRT3 RNA.

The methods of the present invention may be used to treat, prevent or diagnose any cancerous or pre-cancerous tumor. In certain embodiments, the cancerous tumor has a highly glycolytic phenotype. In certain embodiments, the tumor has reduced expression of SIRT3 protein or mRNA relative to non-tumor tissue (e.g., a non-tumor tissue of the same tissue type as the tumor). Highly glycolytic tumors may be located, for example, in a tissue selected from brain, colon, urogenital, lung, renal, prostate, pancreas, liver, esophagus, stomach, hematopoietic, breast, thymus, testis, ovarian, skin, bone marrow and/or uterine tissue.

In some embodiments, methods and compositions of the present invention may be used to treat, prevent or diagnose any cancer. Cancers that may treated, prevented or diagnosed by methods and compositions of the invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

All publications, including patents, applications, and GenBank Accession numbers mentioned herein are hereby incorporated by reference in their entirety as if each individual publication or patent was specifically and individually indicated to be incorporated by reference. In case of conflict, the present application, including any definitions herein, will control.

The invention now being generally described, it will be more readily understood by reference to the following examples, which are included merely for purposes of illustration of certain aspects and embodiments of the present invention, and are not intended to limit the invention.

### EXEMPLIFICATION

### Example 1

### The Metabolic Profile of SIRT3 Null Mice

The switch from oxidative to glycolytic metabolism is a hallmark of tumorigenesis. Identifying mitochondrial regulators of this switch is an area of intense investigation. Mitochondrial enzymes involved in central metabolism, including enzymes in the TCA cycle, oxidative phosphorylation (OXPHOS) and fatty acid oxidation are heavily acetylated. It is therefore possible that reversible acetylation of these pathways could contribute to the Warburg effect.

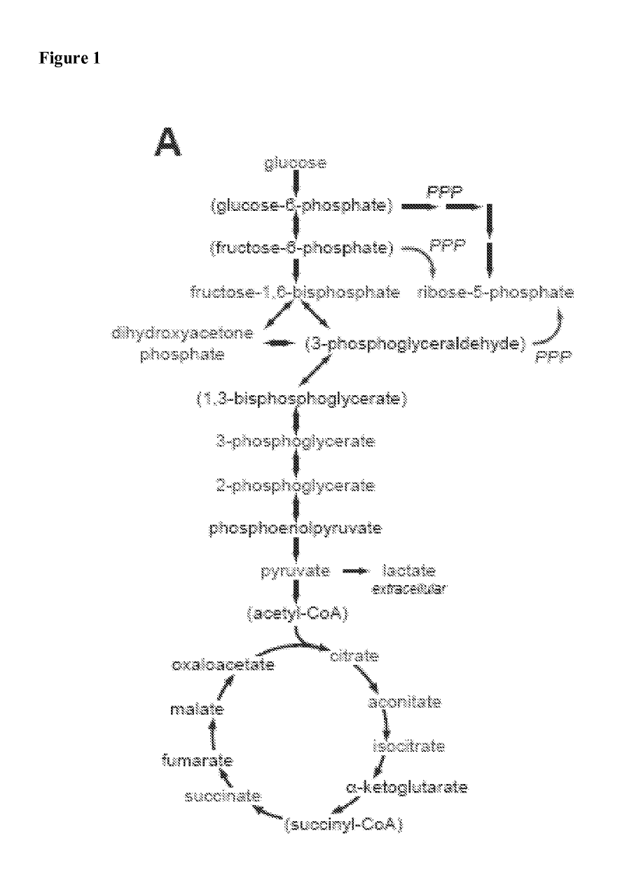

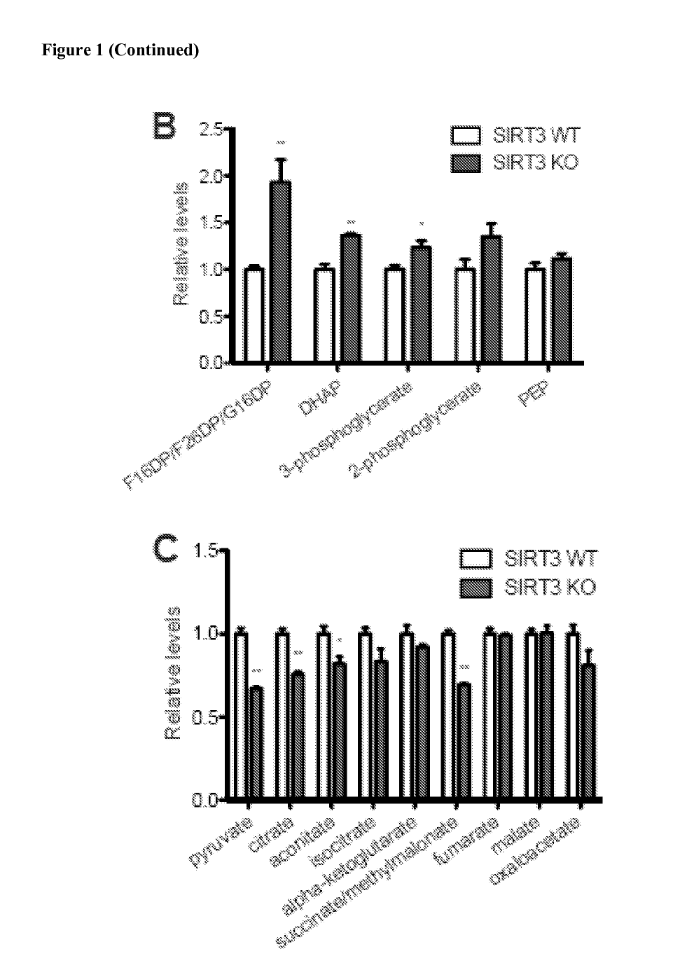

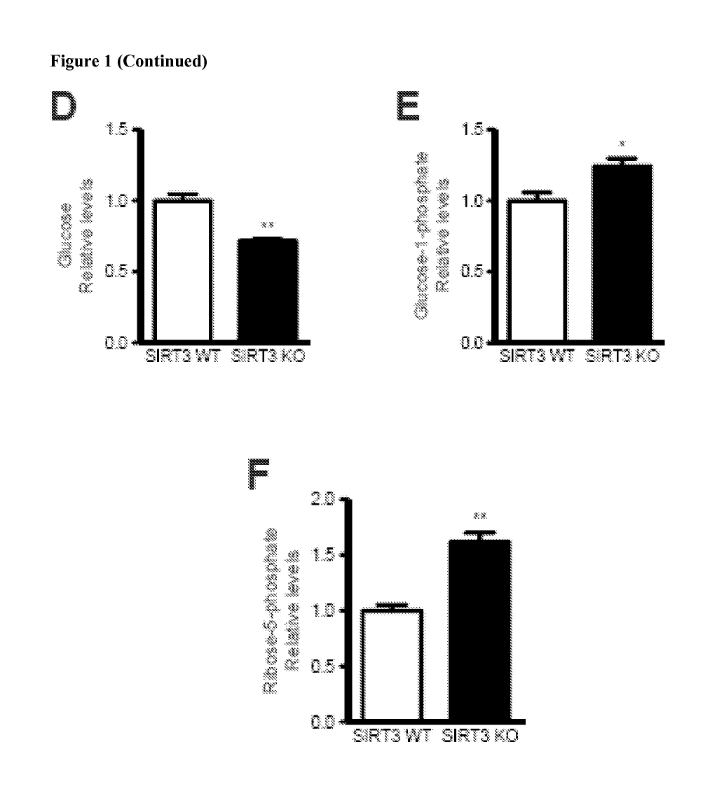

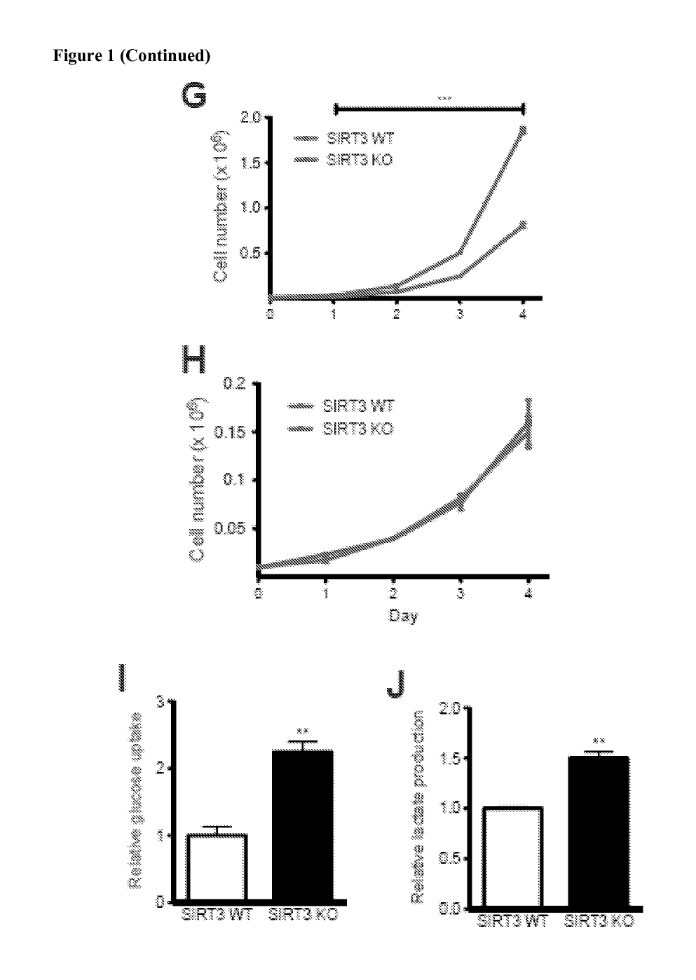

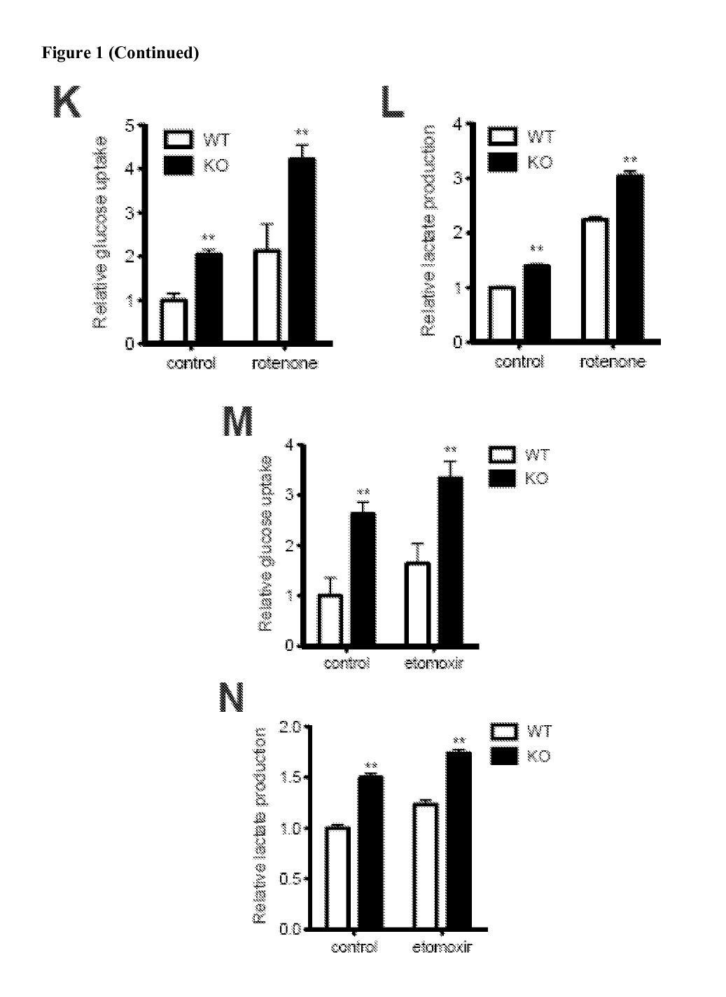

The metabolic profile of SIRT3 null (KO) mouse embryonic fibroblasts (MEFs) was examined. Metabolites were isolated from immortalized SIRT3 WT and KO MEFs (n=4) by methanol extraction and analyzed by liquid chromatography-mass spectrometry (LC-MS). The SIRT3 KO MEFs demonstrated a shift towards glycolytic metabolism when compared to SIRT3 wild-type (WT) MEFs (FIG. 1a), similar to changes that have been reported for cancer cells in culture and in vivo. Intermediates of glycolysis were elevated while TCA cycle metabolites were reduced in SIRT3 KO cells (FIG. 1b, c). Consistent with a pattern of increased glucose usage, SIRT3 KO cells had lower levels of intracellular glucose (FIG. 1d), while levels of glucose-1-phosphate, a product of glycogenolysis, were increased (FIG. 1e). Moreover, ribose-5-phosphate, the product of the pentose phosphate pathway (PPP), was increased by 62% in SIRT3 KO cells (FIG. 1f), indicating that many glucose metabolites were diverted into the PPP in order to provide the NADPH and ribose necessary for fatty acid or nucleic acid synthesis, respectively.

To confirm that the metabolite patterns reflected an increase in glycolysis, lactate produced and secreted from SIRT3 WT and KO MEFs (n=5) was measured and normalized to cell number. It was found that SIRT3 KO MEFs consumed more glucose and extruded more lactate into the extracellular media than WT cells (FIG. 1i and 1j). To test whether this apparent increase in aerobic glycolysis supported an increase in biomass and proliferation, growth rates of the MEFs were measured by seeding SIRT3 WT and KO MEFs in six-well plates at a density of 5,000 cells/well and counting the number of cells in triplicate every 24 hours. It was found that cells lacking SIRT3 proliferated significantly faster than WT cells (FIG. 1g). To test whether this increased growth rate required aerobic metabolism of glucose, cells were grown in media containing galactose instead of glucose, thereby reducing glycolytic flux and forcing the cell to rely on mitochondrial oxidative phosphorylation. Under these conditions, WT and KO cells grew at the same rate, demonstrating that the increased proliferation of KO cells required enhanced glucose catabolism (FIG. 1h).

To test whether SIRT3 upregulates glycolysis as part of a compensatory response due to diminished oxidative capacity, glucose uptake and lactic acid secretion was examined in the presence of a mitochondrial respiratory inhibitor, rotenone, or an inhibitor of mitochondrial fatty acid oxidation, etomoxir. In WT cells, glycolysis is increased in the presence of both rotenone (FIG. 1k and 1l) and etomoxir (FIG. 1m and 1n). Strikingly, glucose uptake and lactate production remain elevated in the SIRT3 KO cells even in the presence of oxidation inhibitors (FIG. 1k-1n). These data demonstrate that upregulated glycolysis in SIRT3 null cells does not result solely from nonspecific compensation for decreased mitochondrial oxidative functions. Instead, these data indicate that, surprisingly, SIRT3 may regulate glycolysis via activation of a specific signaling pathway.

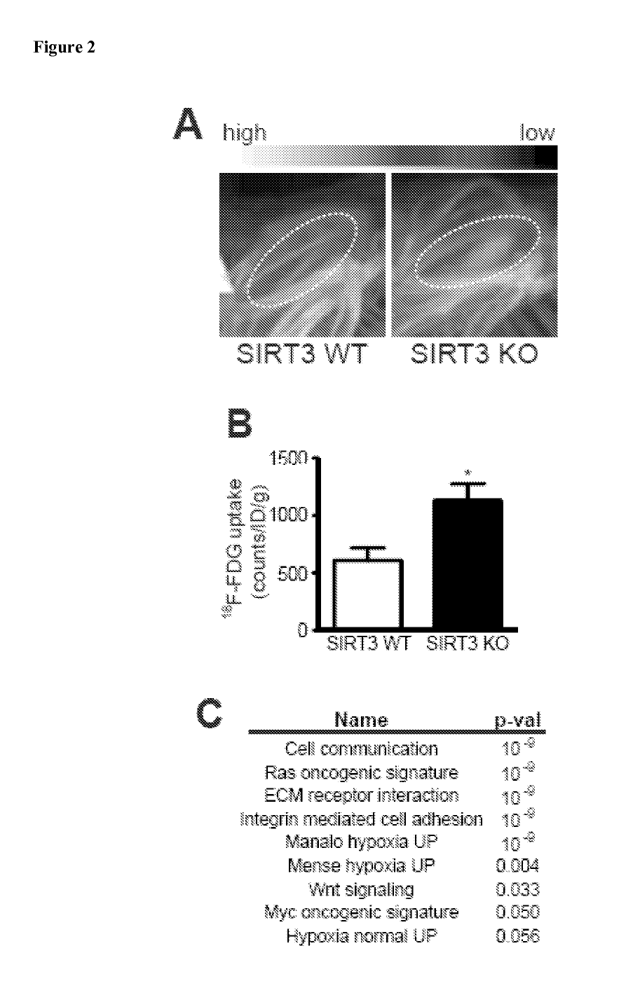

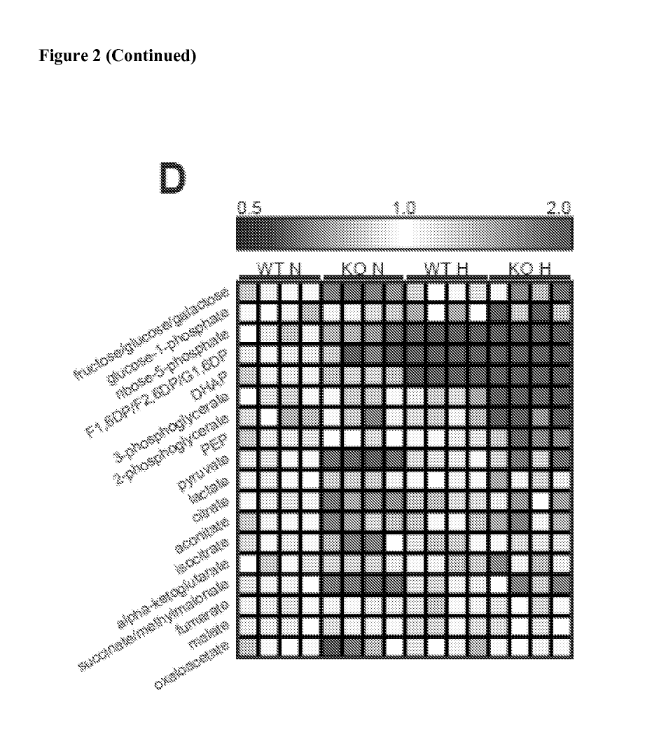

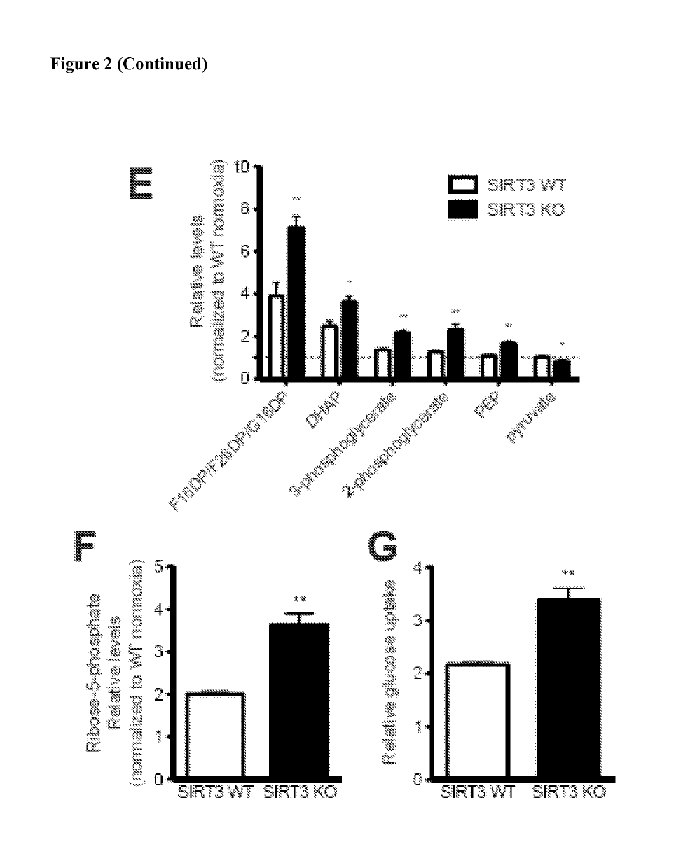

Mice were injected with ¹⁸F-fluordeoxyglucose (¹⁸F-FDG) and scanned using positron emission tomography-computed tomography (PET/CT) in order to monitor glucose uptake. Brown adipose tissue (BAT), which exhibits high glucose uptake, was examined. SIRT3 KO mice had a increased in ¹⁸F-FDG uptake in brown adipose tissue (BAT) compared with WT mice (FIGS. 2a and 2b), even though the mass of BAT in SIRT3 KO mice was not larger than in WT mice. Glucose uptake in BAT is regulated by the β-adrenergic pathway and is thus dramatically increased by cold exposure. ¹⁸F-FDG uptake was measured in BAT of SIRT3 WT and KO after a 6 hour cold challenge and found that SIRT3 KO mice have higher ¹⁸F-FDG uptake both at room temperature and at 4° C. (FIG. 2b), illustrating that SIRT3 WT and KO mice have a similar increase in BAT glucose uptake in response to β-adrenergic signaling. These differences in BAT glucose uptake occur independently of obvious changes in whole-body glucose homeostasis. Changes in blood glucose levels were not detected.

To examine mechanisms underlying increased glucose uptake in SIRT3 KO BAT, genome-wide expression profiling was performed on RNA isolated from BAT and performed gene set enrichment analysis (GSEA) using the ranked gene list from most up- to most down-regulated in SIRT3 KO mice in order to identify the biological pathways most significantly altered by SIRT3 loss. SIRT3 loss up-regulated pathways important in tumorigenesis. Of the nine gene sets most significantly overrepresented in SIRT3 KO BAT, three were independently defined as gene sets induced by exposure to hypoxia (FIG. 2c). Hypoxia itself increases ¹⁸F-FDG uptake and is associated with many transcriptional changes that result in increased glucose uptake and utilization.

The similarity between gene signatures of SIRT3 KO mice and hypoxic cells was particularly notable because hypoxia induces a metabolic shift similar to that caused by loss of SIRT3, including a decrease in mitochondrial substrate oxidation and an increase in glycolysis. To test the role of SIRT3 in hypoxia-induced metabolic reprogramming, metabolites isolated from MEFs cultured at 21% O₂ (normoxia) or 1% O₂ (hypoxia) for 12 hours were analyzed. The increase in glycolytic intermediates caused by hypoxia was similar to the effects of SIRT3 deletion (FIG. 2d). Furthermore, hypoxia and SIRT3 loss had additive effects: while intermediates of glycolysis, glycogenolysis and the PPP were elevated by hypoxia, levels of these metabolites were dramatically higher in SIRT3 KO MEFs under these conditions (FIGS. 2e and 2f). Consistent with the metabolite profiles, hypoxia increased glucose uptake in both cell lines, and SIRT3 KO or knock-down cells consumed even more glucose than control cells (FIG. 2g). Taken together, these data illustrate that SIRT3 loss and hypoxia result in similar metabolic shifts and implicate dysregulated activation of the hypoxia pathway as a cause of the metabolic reprogramming of SIRT3 null cells.

Taken together, these data show that loss of SIRT3 causes metabolic reprogramming to promote the Warburg effect.

### Example 2

### Association of SIRT3 with Mitochondrial Metabolic Enzymes

SIRT3 could regulate the balance between oxidative and glycolytic metabolism by targeting multiple mitochondrial metabolic enzymes. Anti-FLAG immuoprecipitates of HEK293T cells transiently expressing vector, FLAG-tagged SIRT3, SIRT4 or SIRT5 were immunoblotted with either a cocktail recognizing two subunits of complex II (SDHA and SDHB, as labeled) and a subunit of complex V as well as an antibody against SUCLA2, an antibody cocktail recognizing representative subunits of each of the five respiratory complexes (1-V), or an antibody against the FLAG epitope. It was found that SIRT3 specifically interacts with succinyl-CoA synthetase (SUCLA2) and succinate dehydrogenase (SDHA and SDHB), which also represents complex II of OXPHOS (FIG. 2a, top two panels).

Since flux through the TCA cycle serves to produce high-energy electrons to fuel OXPHOS, it is likely that the two pathways would share overlapping regulation. Indeed, it was found that SIRT3 physically associates with the F1α subunit of complex V, in addition to the SDHB subunit of complex II (FIG. 2a, top and middle panels). Furthermore, when anti-FLAG immunoprecipitates of cells expressing vector or FLAG-tagged SIRT1-7 were immunoblotted with antibodies against SDHA, OSCP and FLAG, it was found that, of the seven mammalian sirtuins, only SIRT3 binds SDHA and OSCP, subunits of complex II and V, respectively (FIG. 2b).

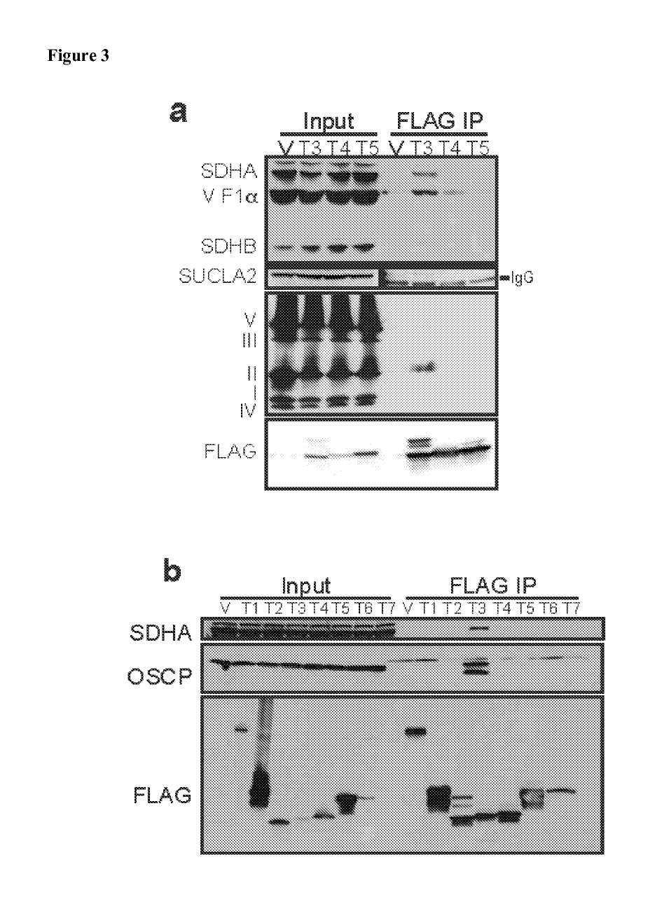

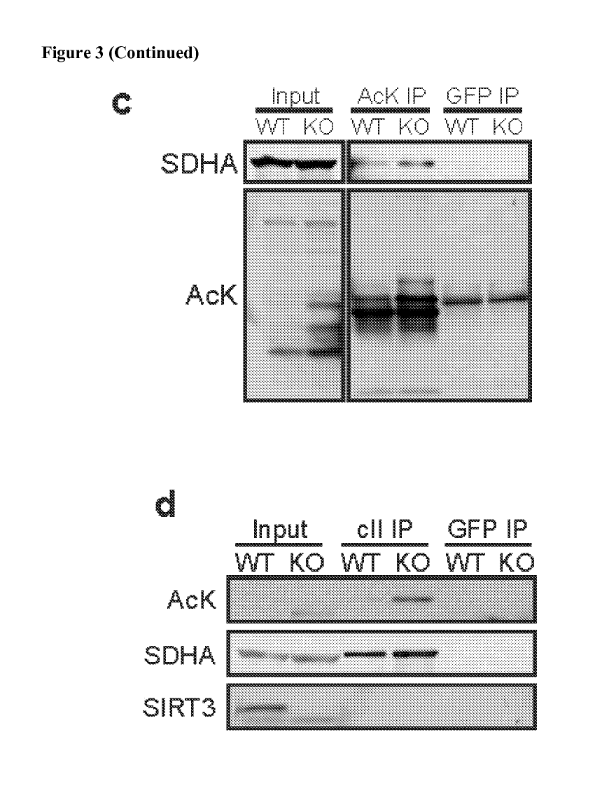

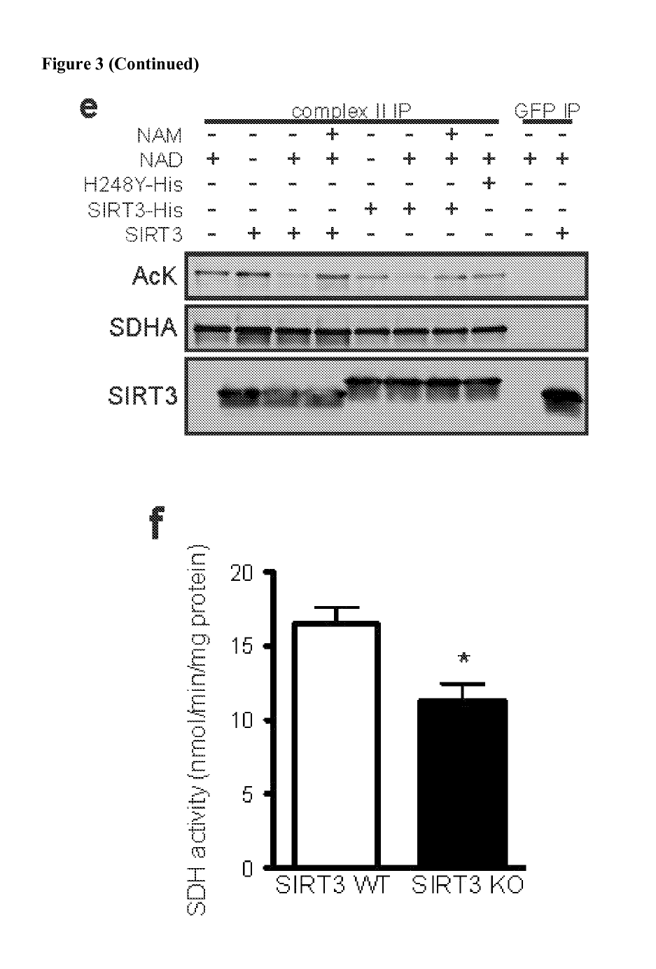

Because SDH holds a unique potential regulatory position at the intersection of the TCA cycle and OXPHOS, the ability of SIRT3 to regulate SDH activity was examined further. It was found that SDHA is acetylated on 13 lysines (FIG. 3). When acetylated proteins and complex II, along with GFP as a negative control, were immunoprecipitated from liver mitochondria isolated from SIRT3 WT or KO mice and probed with antibodies recognizing SDHA, acetyl-lysine (AcK) or SIRT3, it was found that SDHA is hyperacetylated in the absence of SIRT3 (FIG. 2c, d).

Complex II immunoprecipitates were incubated either with recombinant SIRT3 purchased from Biomol, His-tagged SIRT3 (SIRT3-His) or catalytically inactive SIRT3 (SIRT3-H248Y-His) and NAD or nicotinamide (NAM), a sirtuin inhibitor, and immunoblotted for acetyl-lysine (AcK), SDHA and SIRT3. It was found that SIRT3, but not a catalytically inactive mutant, deacetylated SDHA in a NAD-dependent manner (FIG. 2e). Importantly, when malonate-sensitive SDH activity was measured from SIRT3 WT or KO MEFs and normalized to total protein, it was found that SDH activity was reduced by 25% in SIRT3 KO MEFs (FIG. 2f).

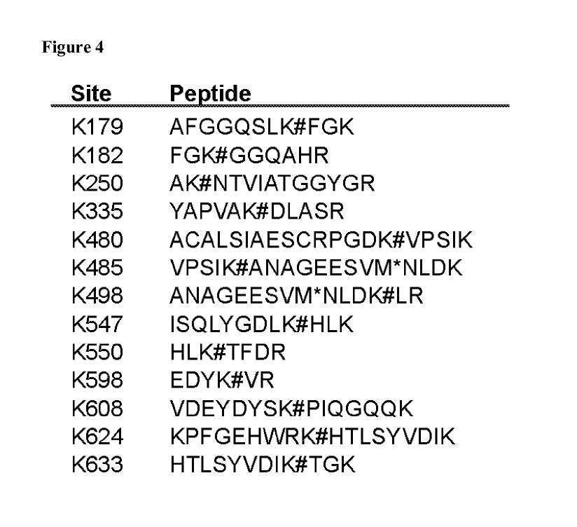

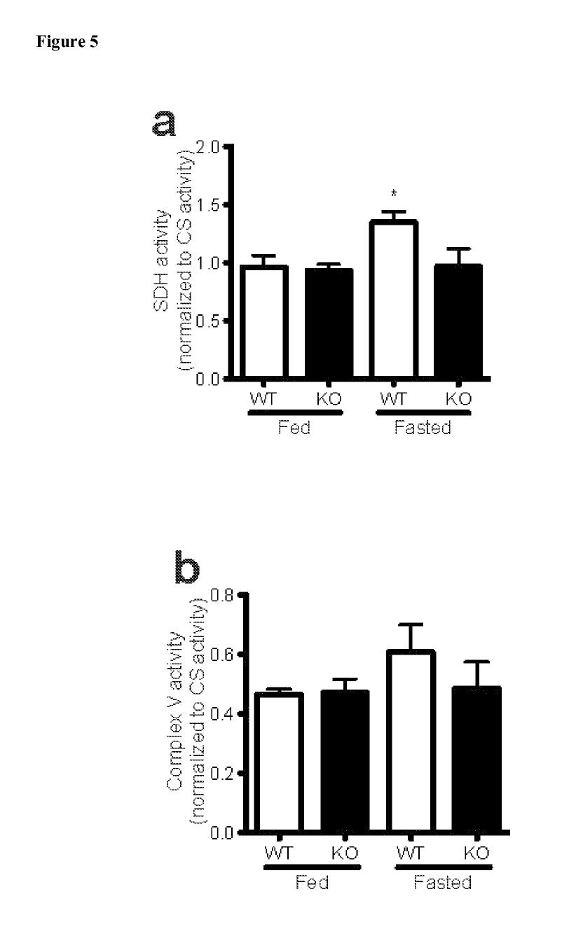

In vivo, SDH activity was reduced in the liver of SIRT3 KO mice. SDH and complex V activity was measured from mitochondria isolated from fed or 48 h fasted SIRT3 WT or KO mice and activity was normalized to citrate synthase activity to control for the mass of functional mitochondria. A similar trend for complex V activity was observed (FIG. 4).

Taken together, these data demonstrate that SIRT3 is required for maximum activation of TCA cycle enzymes and provide a mechanism for the metabolic reprogramming observed in SIRT3 KO MEFs.

### Example 3

### SIRT3 Opposes the Warberg Effect by Destabilizing HIF1α

HIF1, comprised of the heterodimer HIF1α and HIF1β, is the primary driver of increased glycolysis and lactate production during hypoxia. Under conditions of low oxygen, HIF1α is stabilized and promotes transcription of many genes crucial for the cellular response to hypoxia. Consequently, cells lacking HIF1α fail to upregulate glycolytic enzymes and lactic acid production in response to hypoxia.

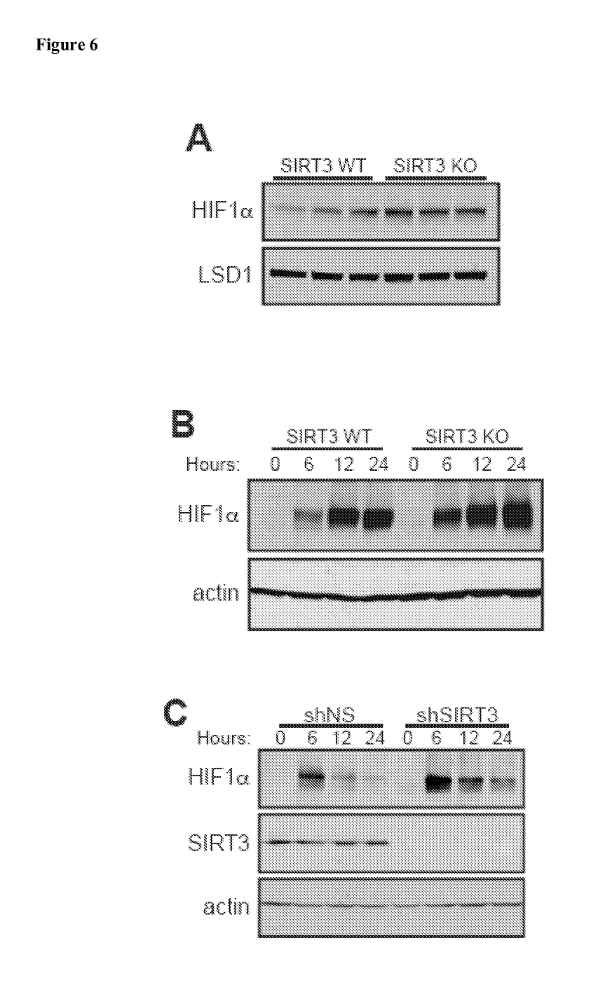

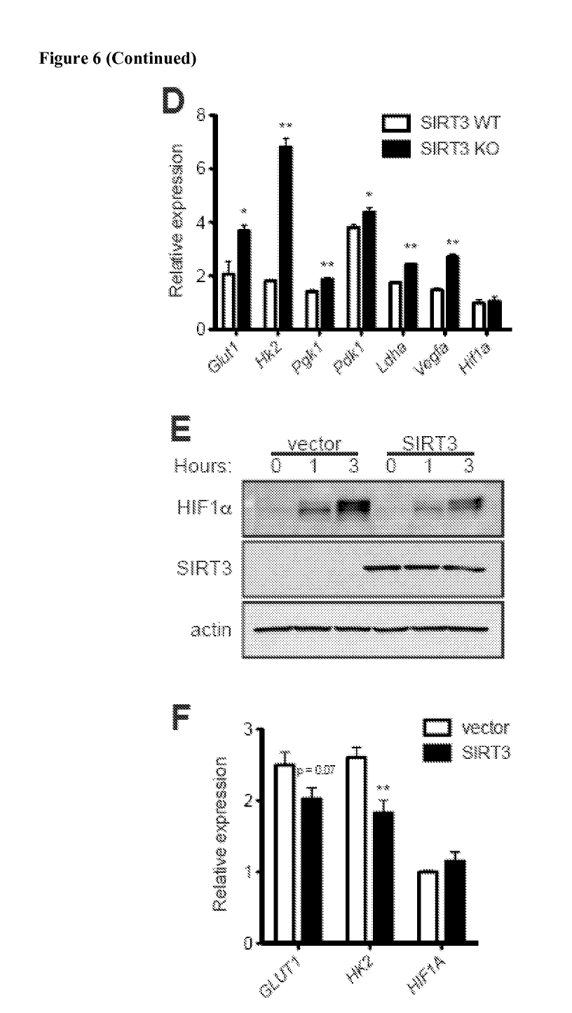

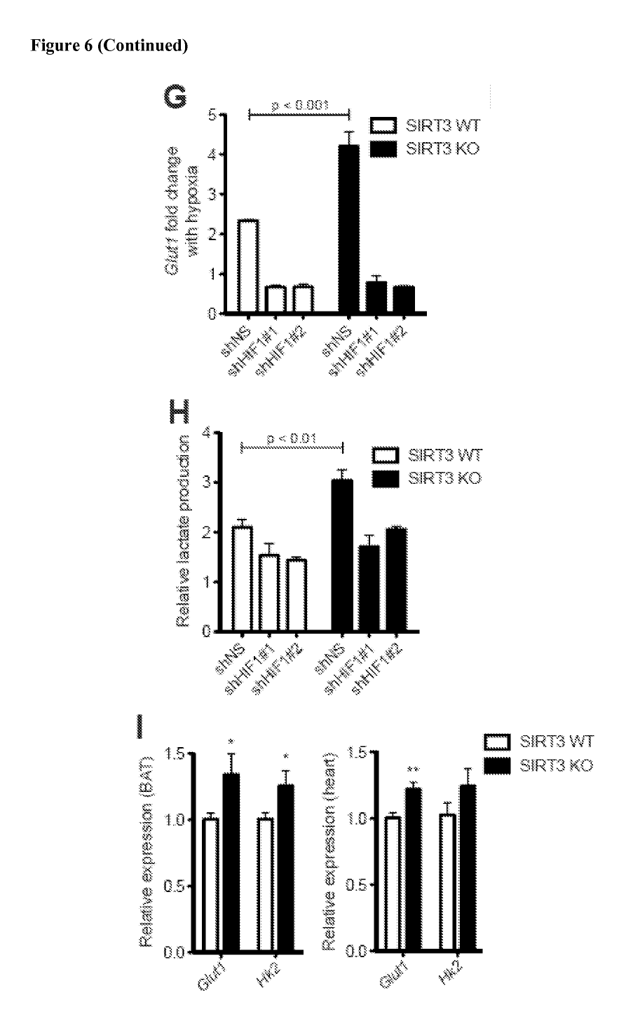

It was investigated whether SIRT3 directly modulates HIF1α stability under normoxic conditions. In the presence of high oxygen, HIF1α is rapidly degraded and difficult to measure from cell lysates, but HIF1α is detectable from isolated nuclei. Indeed, nuclei isolated from SIRT3 deficient cells during normoxia demonstrated elevated levels of HIF1α relative to WT cells (FIG. 6a). Likewise, when MEFs were cultured under 1% O₂, HIF1α was stabilized earlier and to a higher degree in SIRT3 KO cells compared to WT cells in whole cell lysates (FIG. 6b). Comparable results were obtained in HEK293T cells in which SIRT3 expression was stably reduced by lentiviral expression of shRNA against SIRT3 (FIG. 6c). SIRT3 also regulates expression of HIF1α target genes. Both the glucose transporter Glut1 and hexokinase II (Hk2)-HIF1α target genes that are critical for increased glucose uptake and catabolism via aerobic glycolysis or the PPP and are strongly implicated in tumorigenesis were elevated during hypoxia in SIRT3 KO MEFs and SIRT3 knock-down cells relative to control cells (FIG. 6d). Furthermore, the HIF1α targets pyruvate dehydrogenase kinase 1 (Pdk1), lactate dehydrogenase A (Ldha), phosphoglycerate kinase (Pgk1) and vascular endothelial growth factor A (Vegfa) were significantly elevated in SIRT3 KO cells compared to WT cells during hypoxia (FIG. 6d). Similar to the pattern seen in metabolic intermediates of glycolysis, many of these genes were moderately elevated by SIRT3 loss under basal conditions, and SIRT3 deletion and hypoxia had additive effects on expression of HIF1α target genes (FIG. 6d).

To test whether SIRT3 directly represses HIF1α, the levels of HIF1α and its target genes were examined in cells overexpressing SIRT3. SIRT3 overexpression reduced the extent of HIF1α stabilization in hypoxic cells (FIG. 6e). The induction of GLUT1 and HK2 during hypoxia was blunted by SIRT3 overexpression, demonstrating that SIRT3 directly inhibits HIF1α function (FIG. 6f). SIRT3 catalytic activity was required for the full repression of HIF1α target genes: expression of a SIRT3 catalytic mutant did not significantly reduce hypoxic GLUT1 expression. Furthermore, using primary MEFs, it was demonstrated that two SIRT3 KO lines exhibited increased Glut1 expression relative to two WT lines, suggesting that SIRT3 can regulate HIF1α activity in primary cell lines. Taken together, the data show that SIRT3 controls the stabilization of HIF1α and the induction of crucial HIF1α target genes that coordinate aerobic glucose consumption.

The requirement for HIF1α in the glycolytic shift observed in SIRT3 null cells was examined. Two separate shRNA constructs against HIF1α were used to generate SIRT3 WT and KO MEFs with HIF1α levels stably reduced. Normoxic and hypoxic Glut1 expression was measured in these cell lines. Control (shNS) SIRT3 KO MEFs demonstrated an exaggerated response to hypoxia, measured as the fold change in Glut1 expression, compared to control WT MEFs (FIG. 6g). In contrast, WT and SIRT3 KO MEFs expressing either shRNA against HIF1α had comparable responses to hypoxia (FIG. 6g). The increase in lactate production caused by SIRT3 deletion required HIF1α both in normoxia and hypoxia (FIG. 6h). Together, these data demonstrate that SIRT3 regulates aerobic glycolysis through HIF1α.

To probe for evidence of increased HIF1α activation in vivo, levels of HIF1α and HIF1α target genes from tissues of SIRT3 WT and KO mice were measured. Levels of HIF1α protein and many HIF1α target genes involved in glycolysis were significantly elevated in the BAT of SIRT3 KO mice (FIG. 6i). Similarly, several HIF1α target genes showed a trend of increased expression in SIRT3 KO heart (FIG. 6i).

During normoxia, HIF1α is hydroxlylated at two proline residues by a family of oxygen-dependent prolyl hydroxylases (PHD1-3), enabling the tumor suppressor von Hippel-Lindau (VHL) to bind and target HIF1α for ubiquitination and proteasomal degradation. It was tested whether SIRT3 exerted a post-translational effect on HIF1α stability. SIRT1 binds HIF1α and regulates its activity through direct deacetylation. To test whether SIRT3 might act through a similar mechanism, SIRT1 or SIRT3 were immunoprecipitated and probed for interactions with HIF1α. SIRT1, but not SIRT3, pulled down HIF1α.

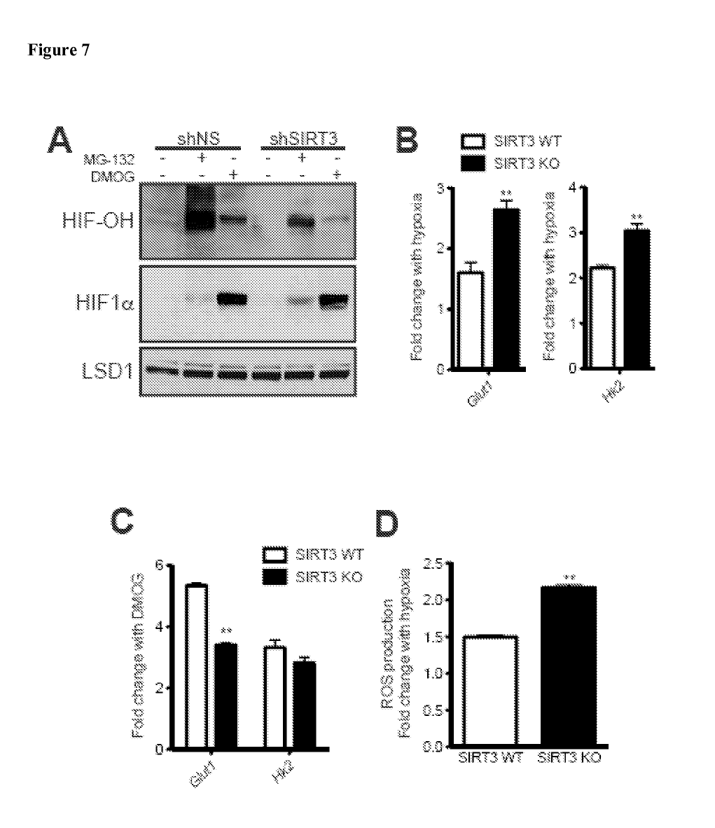

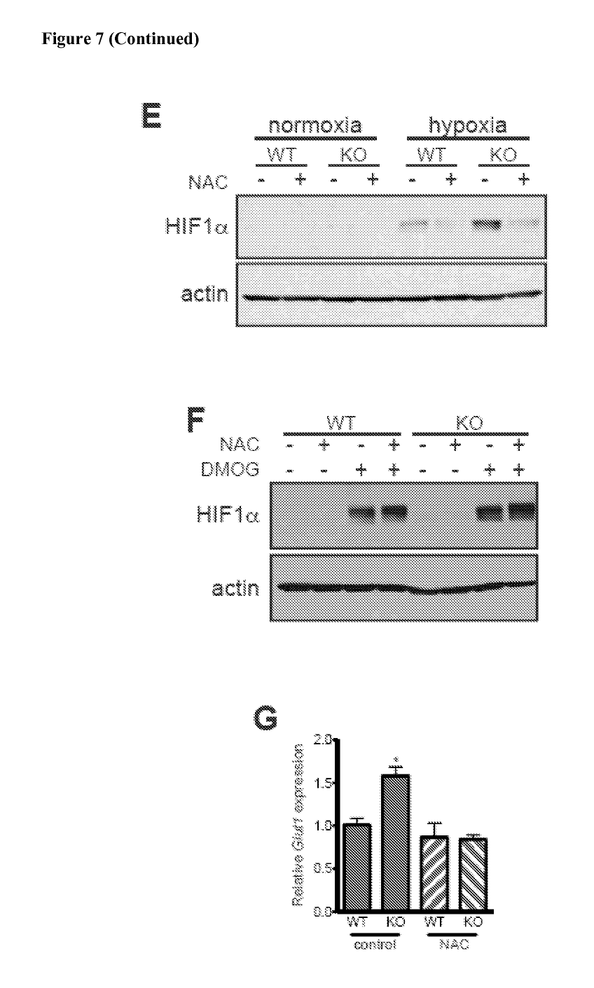

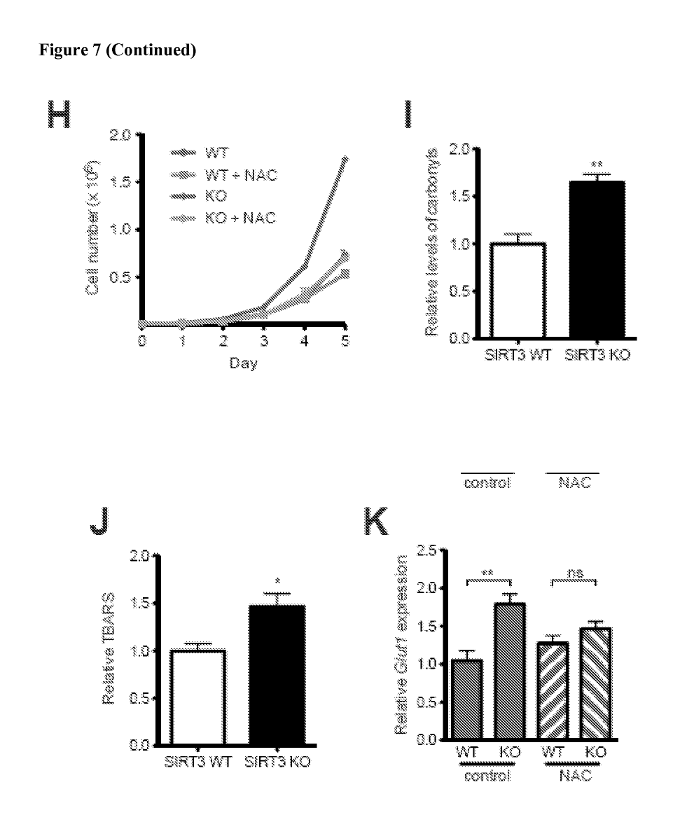

PHD activity was examined in control and SIRT3 knock-down HEK293T cells by treating cells with the proteasomal inhibitor MG-132 (to prevent hydroxylated HIF1α from being degraded) or with DMOG (dimethyloxaloylglycine, to inhibit PHDs). Although SIRT3 knock-down cells accumulated more HIF1αduring MG-132 treatment, they had significantly less hydroxylated HIF1α, indicating that PHD activity is lower in SIRT3 knock-down cells (FIG. 7a). Similarly, SIRT3 WT MEFs demonstrated higher levels of HIF1α hydroxylation than KO MEFs.

A series of experiments were performed comparing the effects of hypoxia and DMOG treatment on SIRT3 WT and KO MEFs. Both hypoxia and DMOG stabilize HIF1α and induce expression of HIF1α target genes (FIGS. 7b and 7c). The relative responses of SIRT3 WT and KO MEFs to hypoxia and DMOG underscore the PHDs as the point of regulation by SIRT3. During hypoxia, HIF1α target genes are induced more strongly in SIRT3 KO cells, illustrating the physiological importance of SIRT3 in regulating the metabolic response to hypoxia (FIG. 7b). In contrast, SIRT3 deletion represses the induction of HIF1α target genes in response to DMOG (FIG. 7c). These data support a model whereby PHD activity is already reduced in SIRT3 KO cells. Consequently, when PHD activity is potently blocked by DMOG, SIRT3 KO cells have a smaller change in PHD activity and thus a smaller induction of HIF1α target genes. Together, these results point to reduced PHD activity as the mechanism of increased HIF1α expression in SIRT3 deficient cells.

Several intracellular signals, in addition to changes in oxygen concentration, are known to regulate PHD activity. Notably, reactive oxygen species (ROS) have been shown to inhibit the PHDs and stabilize HIF1α. Moreover, hypoxia triggers an increase in ROS production that is required for the hypoxic activation of HIF1α. Because SIRT3 is a inhibitor of ROS, increased ROS in SIRT3-deficient cells could contribute to the inhibition of the PHDs. Thus, it was tested whether SIRT3 loss would magnify the increase in ROS associated with hypoxia. The hypoxia-triggered increase in ROS was significantly higher in SIRT3 KO MEFs (FIG. 7d), providing a mechanistic explanation for why SIRT3 null cells have an exaggerated response to hypoxia.

Cells were treated with the anti-oxidant N-acetylcysteine (NAC) in order to probe the model that suppressing ROS could block the effects of SIRT3 deletion. While SIRT3 KO MEFs had higher levels of HIF1α during hypoxia, NAC treatment reduced HIF1α to comparable levels in SIRT3 WT and KO MEFs (FIG. 7E). In contrast, SIRT3 WT and KO MEFs have comparable levels of HIF1α induced by DMOG (FIG. 7F), and NAC could no longer destabilize HIF1α in the presence of DMOG (FIG. 7f). NAC treatment restored Glut1 expression in KO MEFs to WT levels (FIG. 7g). To test whether increased ROS could underlie the proliferative phenotype of SIRT3 KO MEFs, cells were cultured with NAC and growth rates were measured. NAC rescued the increased proliferation of SIRT3 KO MEFs, restoring their growth to WT levels (FIG. 7h). Thus, regulation of ROS by SIRT3 plays an important role in stabilization of HIF1α and activation of glycolytic metabolism in SIRT3 null cells.

To examine the contribution of increased ROS to altered BAT metabolism in vivo, ROS in SIRT3 KO tissues was examined. Two measures of oxidative damage, protein carbonyls and lipid peroxidation, were significantly elevated in SIRT3 KO BAT (FIGS. 7i and 7j). To test whether NAC treatment would reverse the glycolytic signature in SIRT3 KO tissues, mice were treated with NAC for one month and measured expression of HIF1αtarget genes in BAT. NAC repressed expression of HIF1αtarget genes in SIRT3 KO mice, but not in SIRT3 WT mice (FIG. 7k). These data demonstrate that increased ROS production in vivo contribute to enhanced glycolytic gene expression in SIRT3 deficient mice.

### Example 5

### SIRT3 Inhibits Tumor Growth

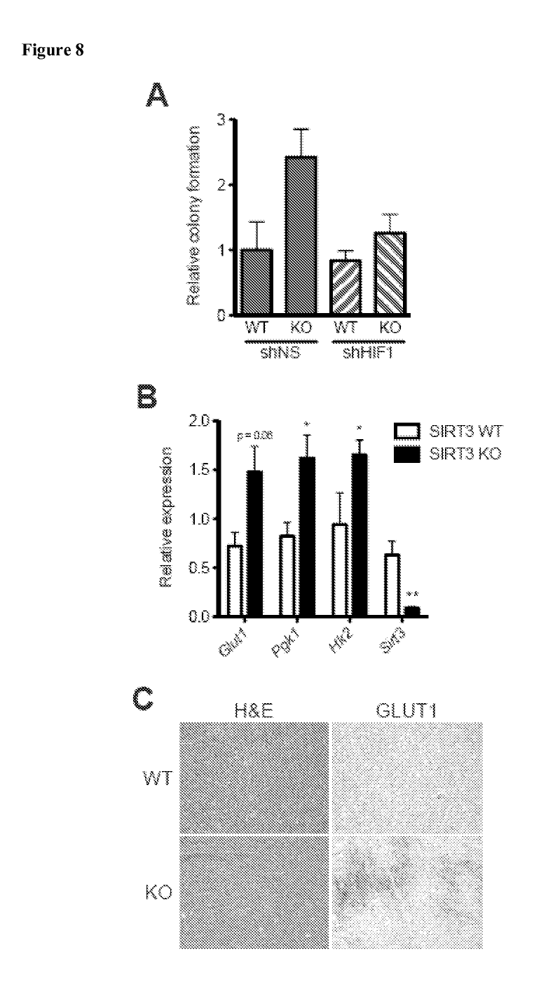

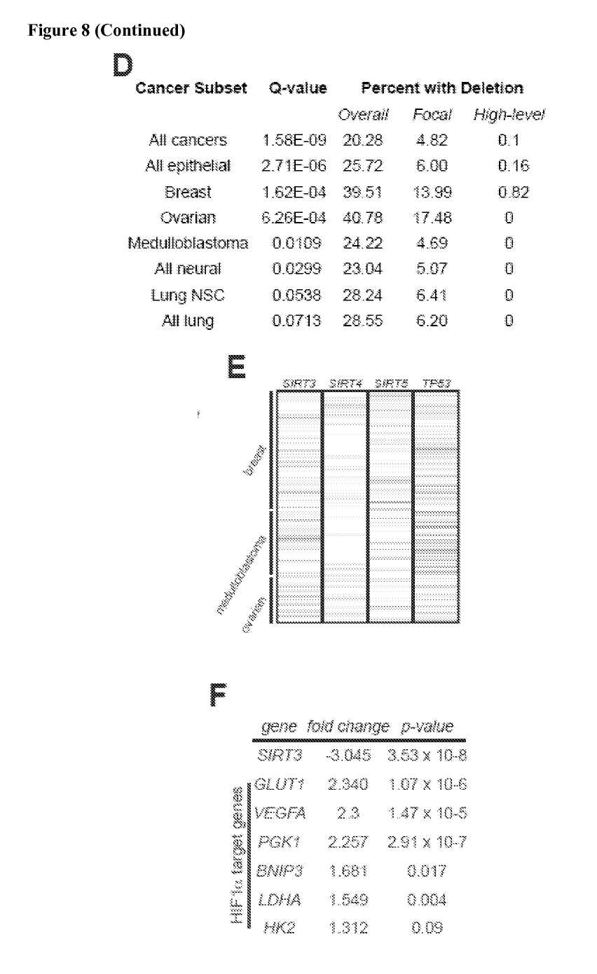

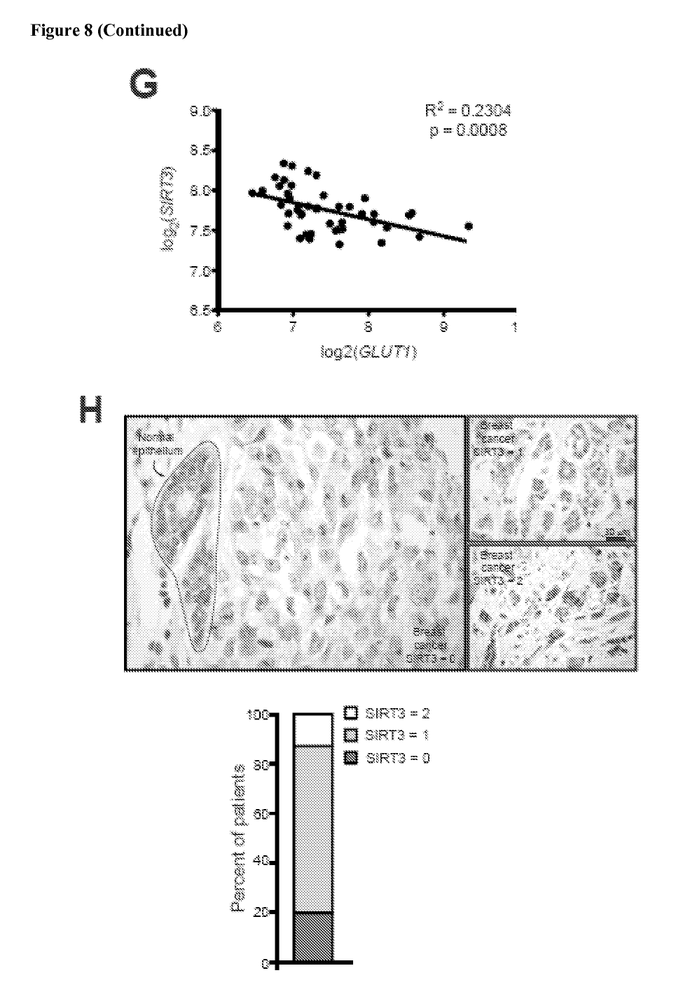

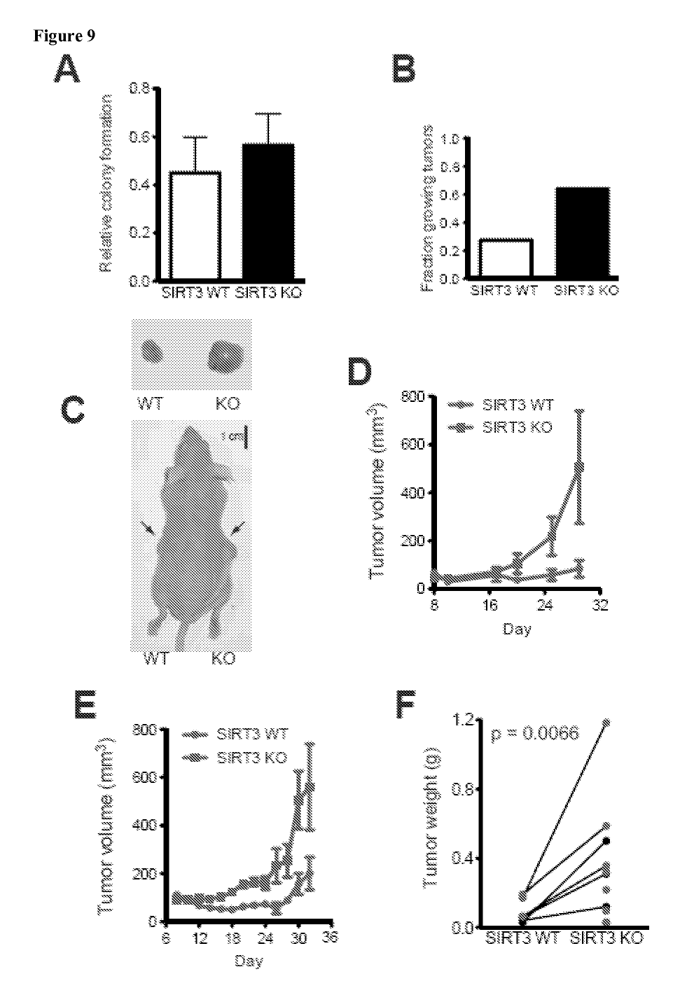

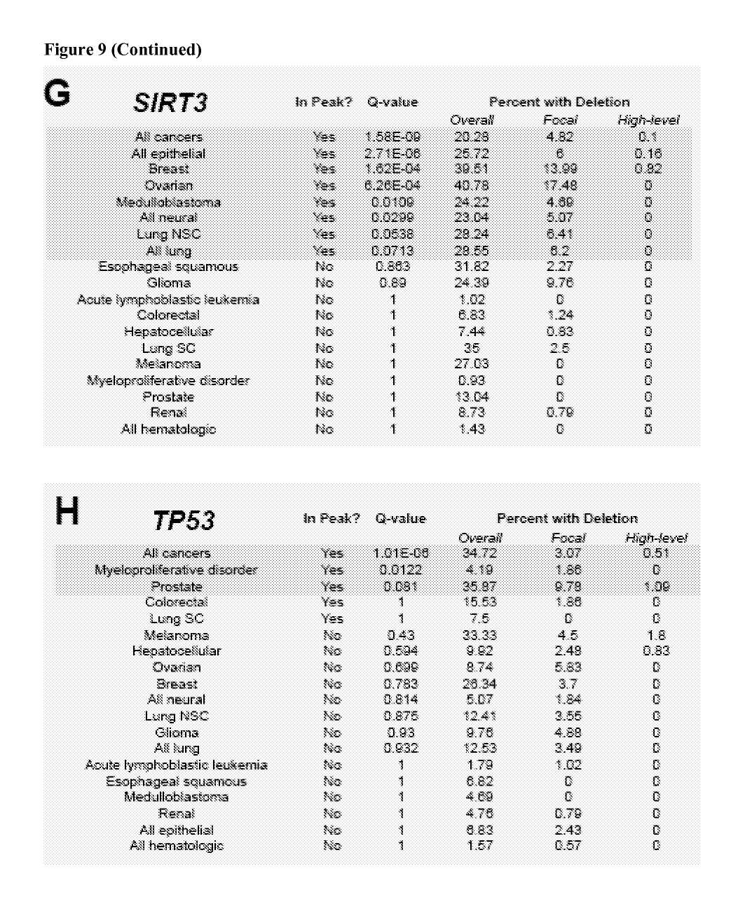

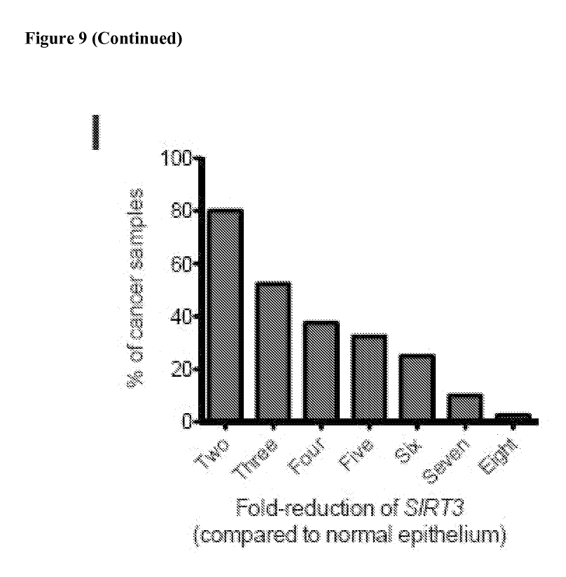

HIF1α activity and aerobic glycolysis are strongly implicated in the Warburg effect. SIRT3 may exert its tumor suppressive activity by opposing the HIF1α-mediated activation of the Warburg effect. Primary MEFs were transformed by expressing the Ras and E1a oncogenes and then HIF1α. was stably knocked down. SIRT3 loss increased colony formation (FIG. 8a). Knock down of HIF1α rescued the increased colony formation of SIRT3 KO cells (FIG. 8A). Furthermore, SIRT3 WT and KO MEFs formed colonies at equivalent rates when cultured in media containing galactose instead of glucose (FIG. 9a), indicating that colony formation required glucose metabolism. Taken together, these data suggest that the metabolic reprogramming mediated by SIRT3 via HIF1α is an important contributor of the tumor-suppressive role of SIRT3.

Xenograft assays were performed with the transformed MEFs in order to probe the metabolic status of SIRT3 null tumors. Tumors lacking SIRT3 had a growth advantage: tumors formed from 64% of KO injections but only 27% of WT injections and tumors lacking SIRT3 grew faster and were bigger than WT tumors (FIG. 9b-f). As tumors are subject to intermittent hypoxia, the expression of rate-limiting glycolytic genes were examined in the xenograft tumors. HIF1α target genes were elevated in SIRT3 KO tumors (FIG. 8b); SIRT3 KO tumors also showed higher levels of GLUT1 protein (FIG. 8c). Taken together, these data suggest that increased levels of glycolytic enzymes, as part of a heightened response to hypoxia, provides a growth advantage for tumor cells lacking SIRT3 in vivo.

### Example 4

### SIRT3 is Deleted in Many Human Cancers

As described above, SIRT3 may regulate tumor cell metabolism and anabolic growth pathways. In order to determine the relevance of SIRT3 in human cancers, the copy-number variations of SIRT3 that are associated with the progression of multiple types of human cancer were examined. Strikingly, at least one copy of the SIRT3 gene is deleted in 20% of all human cancers and 40% of breast and ovarian cancers present in the dataset (FIG. 8d). SIRT3 is significantly focally deleted (deletions of less than a chromosome arm) across all cancers, and focal deletions of SIRT3 were especially frequent in breast and ovarian tumors (FIG. 8d). In contrast, SIRT4 and SIRT5 were not significantly focally deleted in any of the 14 subtypes analyzed (FIG. 8e and data not shown). TP53, a tumor suppressor known to be frequently deleted in many human cancers, is included as a control (FIG. 8e and FIG. 9g-h). Analysis of copy-number changes at the SIRT3 locus revealed no evidence of focal amplifications across 14 types of cancer. Most of the genomic SIRT3 deletions are heterozygous, and SIRT3 deletion frequencies are similar to the well-known breast cancer tumor suppressors, BRCA1 and BRCA2, which are heterozygously deleted in 43% and 40% of human breast cancers, respectively. Intriguingly, the peak region of deletion that includes SIRT3 (11p15.5) does not contain any known tumor suppressor.

Because breast cancers exhibited exceptionally high frequency of SIRT3 deletions compared to other tumor types (FIG. 8d), SIRT3 was further examined in human breast cancers. Elevated HIF1α expression in breast carcinomas is associated with tumor aggressiveness and poor prognosis. Many breast cancer cells exhibit increased glycolysis, and expression of GLUT 1 is a characteristic feature of may breast cancer biopsies. In xenograft models, SIRT3 loss increases expression of HIF1α target genes and results in strong GLUT1 expression (FIGS. 8b and 8c). Gene expression profiling of 7 normal breast samples and 40 ductal breast carcinomas revealed that SIRT3 expression is significantly reduced (p=3.53e⁻⁸) in breast carcinomas (FIG. 80. Moreover, several HIF1α target genes—most notably GLUT1—were significantly increased in the same dataset (FIG. 80. When the correlation between SIRT3 and GLUT1 expression in individual samples from this data set was examined and it was revealed that SIRT3 is significantly inversely correlated with GLUT1 (p=0.0008) (FIG. 8g). These results demonstrate that SIRT3 loss is associated with increased expression of HIF1α target genes in vivo and in human breast cancer and provide a metabolic link between SIRT3 deletion and breast cancer tumorigenesis.

To confirm that SIRT3 expression is reduced in human breast cancers, SIRT3 protein levels were analyzed by immunohistochemistry in normal breast epithelium in addition to a large panel of human breast cancer tissue. Out of 46 patient samples, only 6 demonstrated SIRT3 staining that was positive or as strong as SIRT3 staining in normal epithelium (FIG. 8h). Strikingly, 87% of patients showed decreased or undetectable SIRT3 staining in adjacent cancer tissue and 20% of patients showed no detectable SIRT3 (FIG. 8h). Similarly, gene expression profiling of an independent set of human breast cancer samples revealed that 25% of breast cancers exhibited at least a six-fold reduction in the mRNA of SIRT3 compared to normal breast epithelium (FIG. 9i). This independent dataset provides additional validation for the observation that SIRT3 is deleted in human tumors (FIG. 8d).

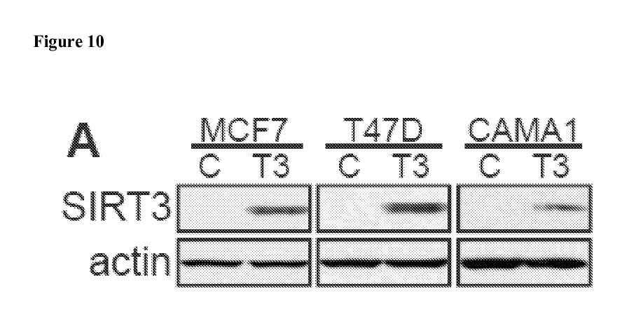

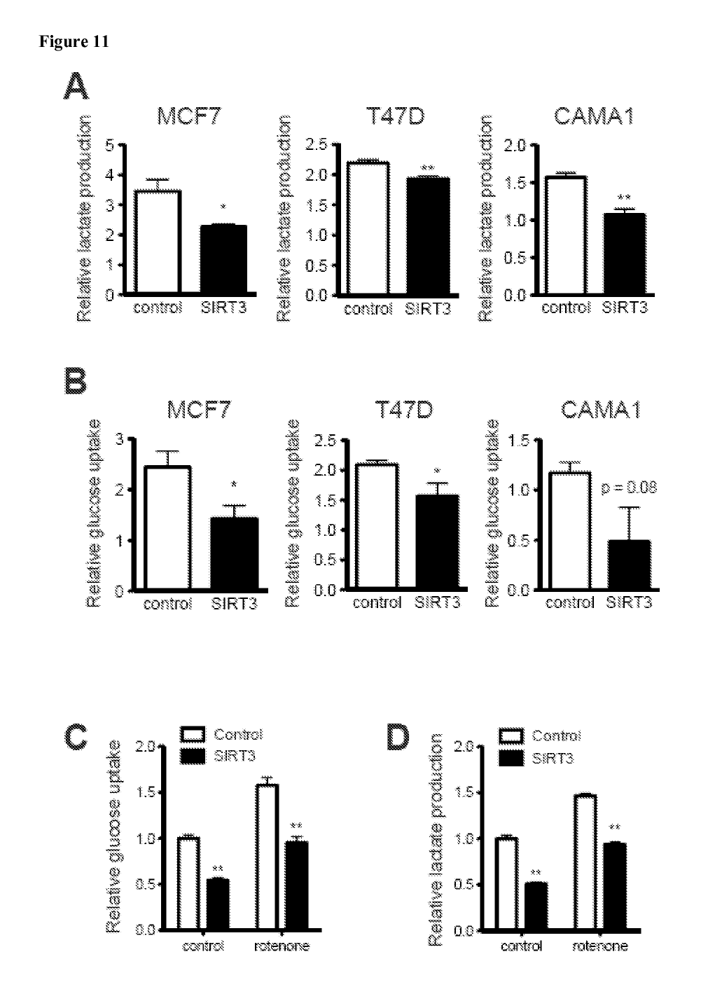

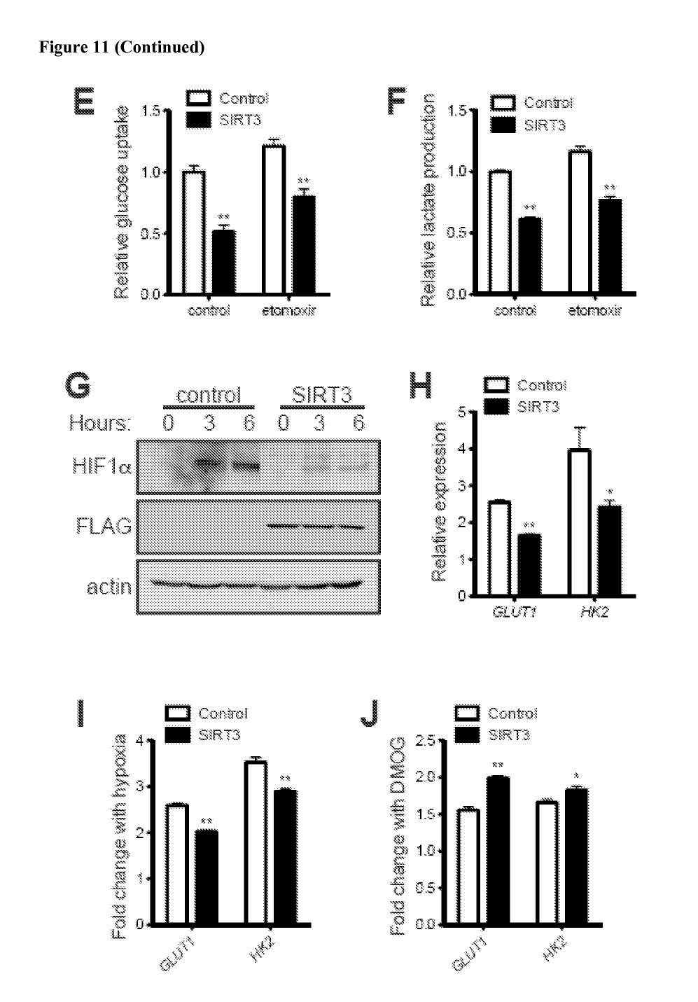

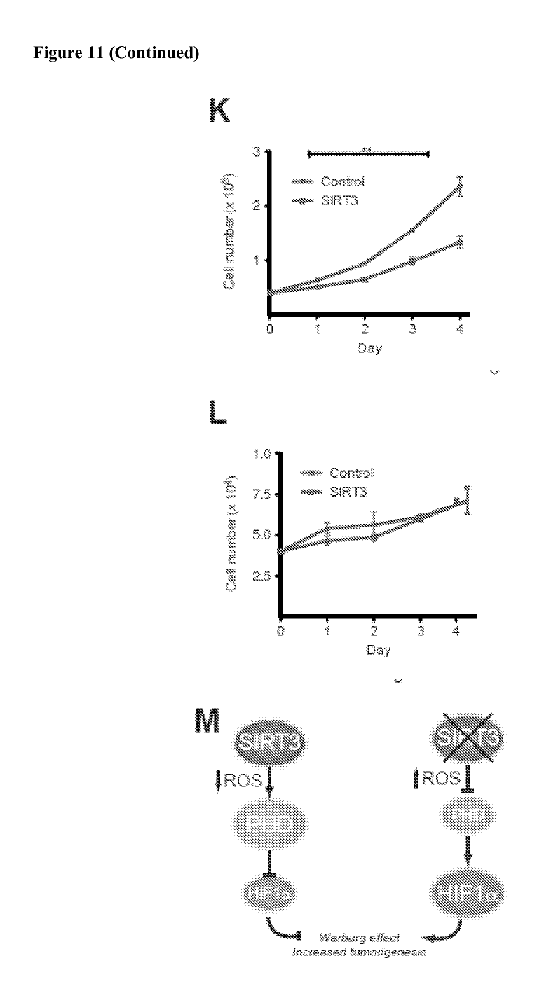

The studies of SIRT3 expression in human cancers indicate that SIRT3 functions as a tumor suppressor in part by preventing the metabolic shift that facilitates tumor growth. In order to examine whether SIRT3 can actively repress the Warburg effect in tumor cells, SIRT3 was stably overexpressed in three independent breast cancer cell lines: MCF7, T47D and CAMA1 (FIG. 10). Glucose uptake and lactate secretion was analyzed in cells during hypoxia in order to simulate the tumor microenvironment. SIRT3 repressed both lactate production and glucose uptake in every cell line tested (FIG. 11a and 11b). These data clearly demonstrate that overexpression of SIRT3 in tumor cells is sufficient to reverse the metabolic shift associated with the Warburg effect.

To examine the contribution of complex I activity or fatty acid oxidation on these phenotypes, glucose uptake and lactate production was measured in the presence of rotenone and etomoxir. Both rotenone and etomoxir increased glucose uptake and lactate production to a similar degree in both control and SIRT3 overexpressing cell lines, indicating that the repression of glycolysis by SIRT3 is independent of the influence of SIRT3 on fatty acid oxidation or complex I activity (FIG. 11c-f.

It was examined whether SIRT3 repressed HIF1α in CAMA1 cells. SIRT3 overexpression strongly reduced HIF1α protein levels and expression of HIF1α target genes in hypoxic cells (FIG. 11g and 11h). Moreover, when the fold change of HIF1αtargets in response to hypoxia or DMOG treatment was examined, it was observed that the inverse of the results using SIRT3 KO MEFs. SIRT3 overexpression blunted the response to hypoxia (FIG. 11i) while increasing the response to DMOG (FIG. 11j). This is consistent with a model of elevated PHD activity in SIRT3 overexpressing cells and illustrates the importance of SIRT3 in regulating the physiological response to hypoxia at the level of the PHDs.

SIRT3-mediated control of glucose metabolism could influence cancer cell proliferation was tested. SIRT3 overexpression significantly repressed proliferation of CAMA1 cells cultured in high glucose (FIG. 11k). Control and SIRT3-expressing cells proliferated at similar rates when cultured in media containing galactose instead of glucose (FIG. 111). These data illustrate that SIRT3 regulates cancer cell growth by influencing the use of glucose for anabolic processes.

### Methods

Metabolites were isolated by methanol extraction from cultured cells and analyzed by liquid chromatography-mass spectrometry (LC-MS). Lactate production was measured using the Lactate Reagent Kit (Trinity Biosciences). For immunoblotting, cell lysates and immunoprecipitations were probed with the following antibodies: Total OXPHOS antibody cocktail, complex II antibody cocktail, and OSCP (Mitosciences); Flag (Sigma); acetyl-lysine, LSD1 and hydroxylated HIF1α (Cell Signaling); SUCLA2 and GFP (Santa Cruz); GDH (USBiological); mouse HIF1α (Novus Biologicals) and human HIF 1 (BD Biosciences). The antibody recognizing murine SIRT3 was raised in rabbits against the (C) DLMQRERGKLDGQDR epitope conjugated to KLH at the added cysteine (Covance Research Products). SDH activity was determined by a colorometric spectrophotometric assay measuring the reduction of 2,6-dichlorophenolindophenol.

For mapping of acetylation sites on SDHA, complex II was immunoprecipitated as described above, run on an SDS-PAGE gel and stained with Coomassie blue. The 70 kDa band corresponding to SDHA was excised and treated with DTT to reduce disulfide bonds and iodoacetamide to derivatize cysteine residues. The protein was then digested in-gel using trypsin. Peptides were analyzed using nano-scale microcapillary C₁₈ liquid chromatography (LC-MS/MS) on an LTQ Orbitrap XL (Thermo Scientific) equipped with an Agilent 1100 binary HPLC pump (Agilent) as previously described. MS/MS spectra were assigned using the SEQUEST algorithm by searching them against a target-decoy protein sequence database based on the mouse IPI database (v 3.60), to which protein sequences of common contaminants were added. Enzyme specificity was set to tryptic; cysteine residues were searched as carbamidomethylated; methionine residues were allowed to be oxidized and lysine residues to be acetylated, and up to 4 missed cleavages on tryptic cleavage sites were accepted. All peptide assignments were filtered to a false-discovery rate of 1%⁸, and MS/MS spectra of acetylated SDHA peptides were manually validated.

Mitochondrial pellets were resuspended to a final concentration of 1 mg/ml in buffer B (280 mM sucrose, 10 mM Tris, pH 7.4). SDH activity was assayed by measuring the phenazine methosulphate (PMS)-mediated reduction of 2,6-dichlorophenolindophenol (DCPIP) of 30 μg of mitochondria at 37° C. in 1.0 mL 50 mM KH₂PO₄, pH 7.5, 1.5 mM KCN, 16 mM succinate, 1 mM PMS, 0.1 mM DCPIP. DCPIP reduction was monitored at 600 nm. For complex V activity, the oxidation of NADH was measured by incubating 5 μg of mitochondria with 0.3 μM NADH, 2 mM phosphoenol pyruvate, 0.5 mM ATP, 5 mM MgCl₂, 10 mM KCl, 50 mM Tris, 5 mg/ml BSA, 0.3 mM FCCP, 0.2 mg/ml antimycin A, 100 U each lactate dehydrogenase and pyruvate kinase. The rate of NADH disappearance was measured at 340 nm. 3 μg/ml oligomycin was added to subtract unspecific activity. For all assays, samples were measured in duplicate and normalized to the rate of citrate synthase activity (measured as the rate of DTNB reduction as described¹) from the sample and experiments were repeated twice with n=3-6.

Fresh media was added to subconfluent cells and 5 μM MitoSOX (Molecular Probes, Invitrogen), 100 μM MnTBAP (Calbiochem) and 100 μM Antimycin A (Sigma) were added where indicated. After 30 minutes, cells were trypsinized and washed twice with Hank's buffered salt solution (HBSS) (GIBCO, Invitrogen) and resuspended at a concentration of 5×10⁶ cells/ml in HBSS with 1% BSA. Samples were analyzed by flow cytometry with 488 nm excitation to measure oxidized MitoSox Red. 10,000 cells were counted per sample and mean FL2 intensity was analyzed.

### EQUIVALENTS

Those skilled in the art will recognize, or be able to ascertain using no more than routine experimentation, many equivalents of the specific embodiments of the invention described herein. Such equivalents are intended to be encompassed by the following claims.

We claim:

1. A method of reducing glycolysis in a cell comprising contacting said cell with an agent that increases the activity or expression of SIRT3 in said cell.

2. The method of claim 1, wherein said agent is a small molecule.

3. The method of claim 2, wherein said small molecule increases the activity of SIRT3.

4. The method of claim 1, wherein said agent is a polypeptide.

5. The method of claim 4, wherein said polypeptide is a SIRT3 protein or a fragment thereof.

6. The method of claim 1, wherein said agent is a polynucleotide.

7. The method of claim 6, wherein said polynucleotide encodes a SIRT3 protein or a fragment thereof.

8. The method of claim 1, wherein said cell is a cancer cell.

9. The method of claim 8, wherein said cell has a highly glycolytic phenotype.

10. The method of claim 8, wherein said cell expresses elevated levels of HIF1α.

11. A method of increasing glycolysis in a cell comprising contacting said cell with an agent that decreases the activity or expression of SIRT3 in said cell.

12. The method of claim 11, wherein said agent is a small molecule.

13. The method of claim 11, wherein said agent is a peptide.

14. The method of claim 11, wherein said agent is an inhibitory polynucleotide specific for SIRT3.

15. The method of claim 14, wherein said inhibitory polynucleotide is selected from the group consisting of siRNA, shRNA, and an antisense RNA molecule, or a polynucleotide that encodes a molecule selected from the group consisting of siRNA, shRNA, and/or an antisense RNA molecule.

16. The method of claim 11, wherein said cell has been exposed to a hypoxic environment.

17. The method of claim 11, wherein said cell is a neuron, a cardiac myocyte or a skeletal myocyte.

18-66. (canceled)

67. A method of determining the metabolic phenotype of a tumor cell comprising the steps of:
a) providing a tumor cell; and
b) determining the level of SIRT3 protein or RNA in the tumor cell;
wherein a decreased level of SIRT3 protein or RNA in the tumor cell relative to the level of SIRT3 protein or RNA in a non-tumor cell indicates that the tumor cell has a glycolytic phenotype.

68-69. (canceled)

70. The method of claim 67, further comprising the step of obtaining the tumor cell from a patient.

71. The method of claim 70, further comprising the step of administering an agent that increases the expression or activity of SIRT3 to the patient.
